(19) United States
(12) Patent Application Publication
(10) Pub. No.: US 2016/0243168 A1
(43) Pub. Date: Aug. 25, 2016

(54) ADOPTIVE CELL TRANSFER METHODS

(71) Applicant: Cantex Pharmaceuticals, Inc., Weston, FL (US)

(72) Inventor: Stephen Marcus, Weston, FL (US)

(21) Appl. No.: 15/044,646

(22) Filed: Feb. 16, 2016

Related U.S. Application Data
(60) Provisional application No. 62/200,504, filed on Aug. 3, 2015.
(60) Provisional application No. 62/117,417, filed on Feb. 17, 2015.

(51) Int. Cl. A61K 35/28; A61K 31/727; A61K 35/17; C12N 5/0789

(52) U.S. Cl. CPC A61K 35/28; C12N 5/0647; A61K 31/727; A61K 35/17

(57) ABSTRACT

Methods and compositions are presented for mobilizing hematopoietic stem cells (HSC) and for mobilizing T lymphocytes from the bone marrow and other compartments; use of the mobilized HSCs for HSC transplantation and mobilized T lymphocytes for T cell immunotherapy; for enhancing engraftment of transplanted HSCs and transferred T cells; and for enhancing recovery of hematopoietic system function following HSC transplantation or for enhancing engraftment and/or persistence of T cells following transfer for T cell immunotherapy.

### 1. CROSS-REFERENCE TO RELATED APPLICATIONS

This application claims priority to U.S. Provisional Patent Application Nos. 62/200,504, filed Aug. 3, 2015, and 62/117,417, filed Feb. 17, 2015, the disclosures of which are incorporated herein by reference in their entireties for all purposes.

### 2. BACKGROUND

Adoptive cell transfer (“ACT”) refers to the infusion into patients of autologous or allogeneic cells of various hematopoietic lineages to treat disease.

Hematopoietic stem cell (“HSC”) transplantation, one category of ACT methods, involves the infusion of autologous or allogeneic stem cells to reestablish hematopoietic function in patients whose bone marrow or immune system is damaged or defective. It also allows the introduction of genetically modified HSCs, for example to treat congenital genetic diseases. In typical HSC transplantation, the HSCs are obtained from the bone marrow, peripheral blood or umbilical cord blood.

The use of peripheral blood is preferred for most autologous transplantations and a significant proportion of allogeneic transplantations because of higher stem cell and progenitor cell content as compared to bone marrow or cord blood. Moreover, HSCs obtained from peripheral blood show faster engraftment following transplantation. Because HSCs in the peripheral blood are present at low concentrations, the donor is typically treated with a mobilizing agent, such as granulocyte colony stimulating factor (G-CSF) or granulocyte macrophage colony stimulating factor (GM-CSF), which affects adhesion of HSCs to the bone marrow environment and releases them into the peripheral blood (Cutler et al., Stem Cells 19(2):108-17 (2001)). G-CSF and GM-CSF have side effects such as bone pain, malaise, headache, chills, and fever. In addition, G-CSF is ineffective in about 20% of donors. An alternative mobilizing agent is plerixafor (Mozobil®); however, the approved use for plerixafor is in combination with G-CSF. Side effects of plerixafor include nausea and diarrhea.

T cell immunotherapy, another category of ACT methods, involves the infusion of autologous or allogeneic T lymphocytes that are selected and/or engineered ex vivo to target 32457/33099/DOCS/3922048.3 specific antigens, typically tumor-associated antigens. The T lymphocytes are typically obtained from the peripheral blood of the donor by leukapheresis. In some T cell immunotherapy methods, the T lymphocytes obtained from the donor, such as tumor infiltrating lymphocytes (“TIL”s), are expanded in culture and selected for antigen specificity without altering their native specificity (Stevanovic et al., J. Clin. Oncol., EPub ahead of print, 10.1200/JCO.2014.58.9093 (2015); Dudley et al., J. Clin. Oncol. 23(10):2346-2357 (2005)). In other T cell immunotherapy methods, T lymphocytes obtained from the donor are engineered ex vivo, typically by transduction with viral expression vectors, to express chimeric antigen receptors (“CAR”s) of predetermined specificity. CARs typically include an extracellular domain, such as the binding domain from a scFv, that confers specificity for a desired antigen; a transmembrane domain; and one or more intracellular domains that trigger T-cell effector functions, such as the intracellular domain from CD3ζ or FcRγ, and, optionally, one or more co-stimulatory domains drawn, e.g., from CD28 and/or 4-1BB (Jensen and Riddell, Immunological Reviews 257:127-144 (2014)). In still other T cell immunotherapy methods, T lymphocytes obtained from the donor are engineered ex vivo, typically by transduction with viral expression vectors, to express T cell receptors (“TCR”s) that confer desired specificity for antigen presented in the context of specific HLA alleles (Liddy et al., Nat. Med. 18(6):980-988 (2012)).

In T cell immunotherapy methods, T cells are typically obtained from the peripheral blood of the donor. It is often desirable to obtain as many T cells as possible from the donor, in order to increase the likelihood of obtaining T lymphocytes of desired antigen specificity and/or phenotype (Jensen and Riddell, Immunological Reviews 257:127-144 (2014)). Accordingly, the donor may be treated with a mobilizing agent in order to effect release of T cells resident in the bone marrow and other physiological niches into the peripheral circulation. Mobilizing agents in current use have undesirable toxicities and side effects.

In adoptive cell transfer methods, it is often desired that the infused cells persist in the recipient. In HSC transplantation, for example, the HSCs are typically intended to reconstitute a functioning hematopoietic system for the lifetime of the recipient. In T cell immunotherapy methods, persistence of the transfused T cells allows the T cell effector functions, e.g., targeting and lysing of targeted tumor cells, to continue over extended periods. To increase the likelihood that the transferred cells will engraft and will persist, recipients in ACT methods are often treated before infusion with myeloablative therapy, or with non-myeloablative but lymphodepleting therapy, including chemotherapy or radiation therapy (Dudley et al., J. Clin. Oncol. 23(10):2346-2357 (2005); Jensen and Riddell, Immunological Reviews 257:127-144 (2014)). Current methods of preparing bone marrow for engraftment have undesired side effects.

There is a need, therefore, for safe and effective alternative compositions and methods for mobilizing hematopoietic cells, such as HSCs and T lymphocytes, to the peripheral blood of donors whose cells will be used in adoptive cell transfer, including HSC transplantation and T cell immunotherapy methods. There is also a need for safe and effective alternative compositions and methods useful in preparing bone marrow to improve engraftment of the infused cells in ACT recipients, including HSC transplantation recipients and T cell immunotherapy recipients.

### 3. SUMMARY

It has now been discovered that certain heparin derivatives (collectively, “heparinoids”) that are capable of inhibiting, reducing, abrogating or otherwise interfering with the binding of CXCL12 to CXCR4 (“CXCL-12 interacting heparinoids”) can affect residence of hematopoietic stem cells (“HSCs”), hematopoietic cancer stem cells, and other hematopoietic cells, such as T lymphocytes, in the bone marrow. The cell mobilizing effect of the CXCL12-interacting heparinoids is useful in mobilizing cells for peripheral harvest from donors and also for preparing or conditioning the bone marrow for engraftment in recipients in adoptive cell transfer methods.

Thus, in a first aspect, methods of mobilizing HSCs are provided. The methods comprise administering to a donor subject a heparin derivative capable of inhibiting, reducing, or abrogating binding of CXCL12 to CXCR4 in an amount effective to mobilize HSCs to peripheral blood and/or peripheral tissues. In typical embodiments of the method, the CXCL12-interacting heparinoid is administered in an amount effective to mobilize HSCs from the bone marrow to the peripheral blood and/or peripheral tissue; and then HSCs are isolated therefrom. HSCs isolated from peripheral blood and/or peripheral tissue following mobilization with the CXCL12-interacting heparinoid can be used for transplantation into subjects in need of HSC transplantation.

In another aspect, methods of conditioning a recipient subject in need of HSC transplantation to enhance engraftment of transplanted HSCs are provided. In typical embodiments, the method comprises administering to a subject in need of HSC transplantation a heparin derivative capable of inhibiting, reducing, or abrogating binding of CXCL12 to CXCR4 in an amount effective to enhance engraftment of transplanted HSCs. In various embodiments, the CXCL12-interacting heparinoid is administered to the recipient subject at a time effective to enhance engraftment of transplanted HSCs.

In a further aspect, methods of enhancing recovery of the hematopoietic system in a recipient of HSC transplantation are provided. The method comprises administering to a subject who has received transplanted HSCs a heparin derivative capable of inhibiting, reducing, or abrogating binding of CXCL12 to CXCR4 in an amount effective to enhance recovery of the hematopoietic system of the subject. In various embodiments, enhancing recovery of the hematopoietic system includes enhancing recovery of myeloid and/or lymphocytic function. In various embodiments, the CXCL12-interacting heparinoid is administered to the subject at a time effective to enhance recovery of the hematopoietic system function following HSC transplantation.

In an additional aspect, methods are presented for obtaining T lymphocytes for T cell immunotherapy. The methods comprise administering to a donor subject a CXCL12-interacting heparinoid in an amount effective to increase in the peripheral blood of the donor the concentration of T lymphocytes having at least one desired phenotype; and obtaining T lymphocytes from the donor's peripheral blood. In certain embodiments, the at least one desired phenotype is selected from the group consisting of:

CD4⁺;

CD8⁺;

CD45RA⁺, CD62L⁺, CD28⁺, CD95⁻;

CD45RA⁺, CD62L⁺, CD28⁺, CD95⁺;

CD45RO⁺, CD62L⁺, CD28⁺, CD95⁺;

CD45RO⁺, CD62L⁻, CD28⁺;

CD45RO⁺, CD62L⁻, CD28⁺, CD95⁺;

CD45RO⁺, CD62L⁻, CD28⁺, Perforin^{hi}, Gzmb^{hi}; and

CD45RO⁺, CD62L⁻, CD28⁺, CD95⁺, Perforin^{hi}, Gzmb^{hi}.

In particular embodiments, the T lymphocytes so obtained are then transduced ex vivo with at least one expression vector.

In a further aspect, methods are presented for conditioning a T cell immunotherapy recipient to enhance the establishment or engrafting of donor T cells in the recipient, comprising: administering a CXCL12-interacting heparinoid to the recipient in an amount and for a period sufficient to deplete the recipient's bone marrow of cellular elements, at a time when depleting the recipient's bone marrow of cellular elements enhances establishment or engraftment of donor T lymphocytes in the recipient.

### 4. BRIEF DESCRIPTION OF THE DRAWINGS

FIG. 1 shows the chemical formula of the ATIII-binding pentasaccharide sequence of USP heparin (also known as “unfractionated heparin”, or “UFH”) and the comparable sequence of a 2-O, 3-O-desulfated heparin derivative (“ODSH”) prepared by cold alkaline hydrolysis of UFH.

FIGS. 2A-2C are photomicrographs of serial bone marrow biopsies of a patient with acute myeloid leukemia (“AML”, also known as acute myelogenous leukemia) treated with an ODSH pharmaceutical composition (“CX-01”) in combination with cytarabine and idarubicin, as described in Example 1. FIG. 2A shows bone marrow prior to treatment. FIG. 2B shows marrow at day 14 of treatment. FIG. 2C shows day 28 marrow.

FIG. 3 shows the concentration-dependent inhibition of CXCR4 binding to SDF1α (CXCL12) by ODSH in an in vitro binding assay.

### 5. DETAILED DESCRIPTION

### 5.1. Overview of Experimental Observations

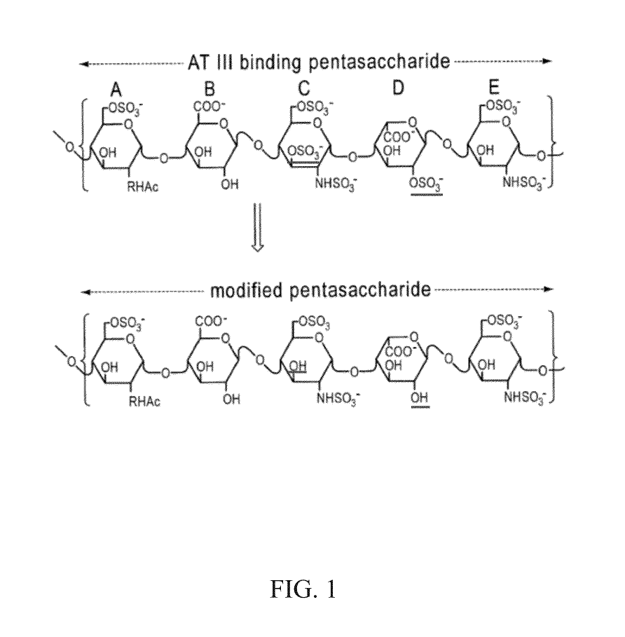

A pharmaceutical composition comprising the substantially non-anticoagulating 2-O, 3-O desulfated heparin derivative, ODSH (see FIG. 1), was previously found to attenuate the myelosuppressive side effects of the chemotherapy regimen used to treat metastatic pancreatic cancer in a human clinical trial (see U.S. Pat. No. 8,734,804, incorporated herein by reference in its entirety). To confirm the myeloprotective effects of ODSH, a second clinical trial was initiated in a different cancer, acute myeloid leukemia (“AML”, also known as acute myelogenous leukemia), treated with a different myelosuppressive chemotherapeutic regimen, idarubicin plus cytarabine (ClinicalTrials.gov identifier: NCT02056782).

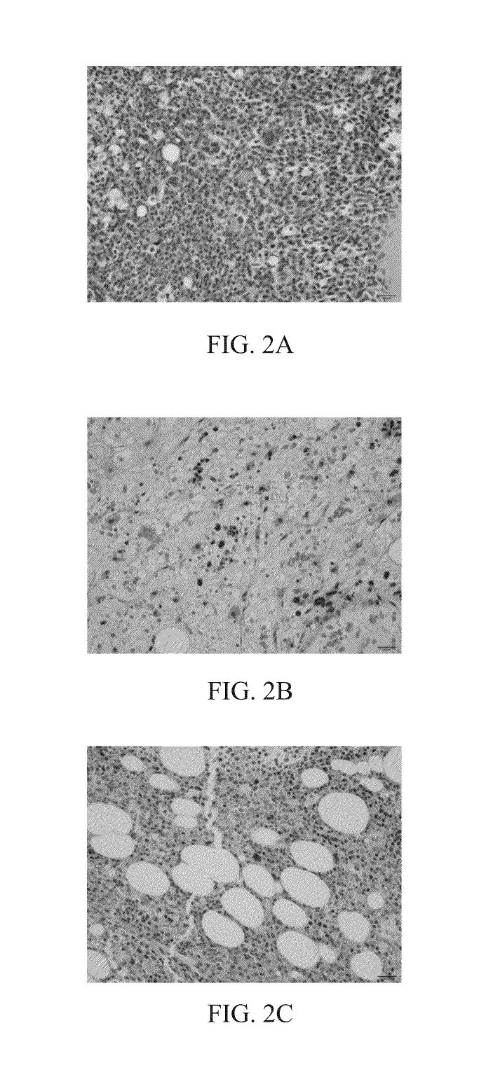

As described in detail in Example 1, below, ODSH significantly attenuated the myelosuppressive side effects of the idarubicin+cytarabine anti-AML chemotherapy regimen, as expected. As described in Example 2, serial bone marrow biopsies drawn from one of the patients treated with ODSH, cytarabine, and idarubicin in the AML clinical trial unexpectedly showed significant depletion of cellular elements in addition to the expected depletion of leukemic cells. FIG. 2A shows bone marrow prior to treatment, demonstrating that the marrow is packed with leukemia cells. FIG. 2B shows marrow at Day 14 of the induction cycle, demonstrating elimination of most normal bone marrow cells as well as leukemia cells. FIG. 2C shows day 28 marrow, with no evidence of leukemic cells and restoration of normal bone marrow appearance and function.

The unexpected clearance of non-neoplastic cells from the marrow seen in the Day 14 biopsy demonstrated that ODSH is capable of mobilizing cells from multiple hematopoietic lineages from the marrow into the peripheral circulation.

CXCL12, also known as Stromal Cell Derived Factor-1 or SDF-1, was originally described as a CXC chemokine produced locally within the bone marrow compartment to provide a homing signal for hematopoietic stem cells. CXCL12 is the ligand for the CXCR4 receptor on the surface of HSCs; ligation of CXCR4 by CXCL12 is known to promote stem cell survival, proliferation, migration, and chemotaxis (see, e.g., Lapidot et al., Leukemia 16(10):1992-2003 (2002)). It has also been reported that the CXCR4 receptor is prominently expressed on the cell membrane of many cancer cells, particularly cancer stem cells (Yu et al., Gene 374:174-9 (2006); Cojoc et al., Oncotargets & Therapy 6:1347-1361 (2013)), and that the CXCL12/CXCR4 interaction may mediate migration of cancer cells to anatomic sites that produce CXCL12 (Wald et al., Theranostics 3:26-33 (2013); Cojoc et al., supra).

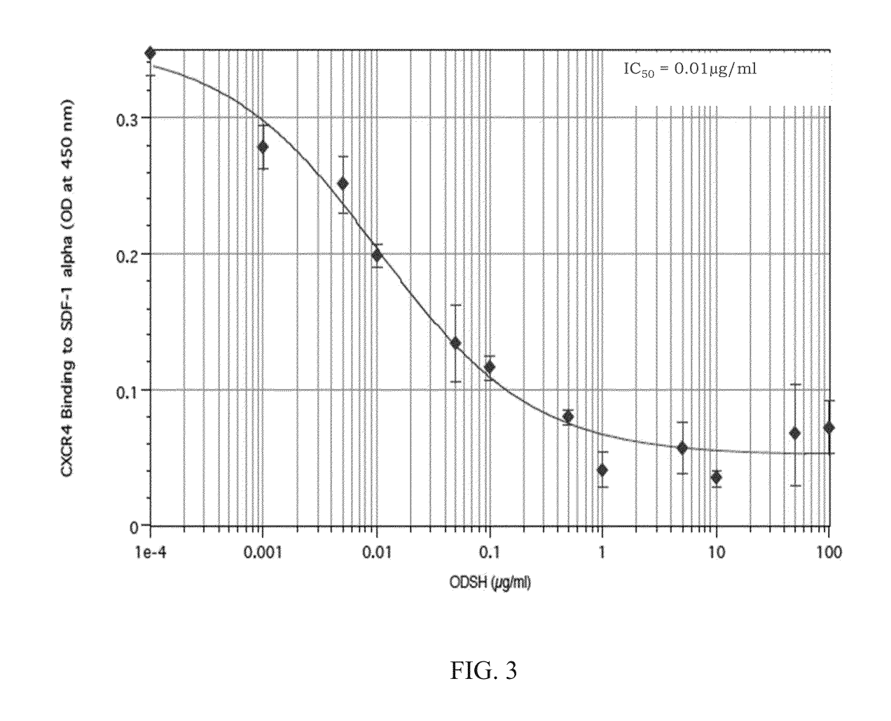

To determine whether the ODSH-mediated mobilization of cells from the bone marrow observed in the AML clinical trial could be attributed to abrogation of or interference with the binding of CXCL12 to CXCR4, an in vitro inhibition assay was performed. Results are reported in Example 3 and FIG. 3.

As shown in FIG. 3, ODSH inhibits binding of CXCL12 (SDF-1) to CXCR4 in a concentration-dependent fashion, with an IC₅₀ of 0.010 μg/ml. This inhibitory concentration is well within the range of plasma concentrations expected to have been achieved in the AML trial: as detailed in Example 2, patients were administered a bolus of 4 mg/kg followed by a continuous intravenous infusion at a dose of 0.25 mg/kg/hr for a total of 7 days; an earlier phase I pharmacokinetics study had demonstrated that a bolus of 8 mg/kg followed by continuous intravenous infusion of 0.64 to 1.39 mg/kg/h provides a maximum mean plasma level of about 170 μg/ml, and steady state concentrations of about 40 μg/mL (Rao et al., Am. J. Physiol. Cell Physiol. 299:C997-C110 (2010)). These concentrations were not significantly anticoagulating.

Because interaction of CXCL12 to CXCR4 is involved in homing and proliferation of HSCs in the bone marrow, these results indicate that ODSH, and heparin derivatives that analogously abrogate or interfere with the binding of CXCL12 to CXCR4, can be used to mobilize HSCs from the bone marrow.

Moreover, the recovery of the marrow by Day 28 seen in FIG. 2C demonstrates further that the ODSH-mediated mobilization of cells from the marrow does not adversely affect the ability of the marrow to repopulate and support multi-lineage hematopoiesis. Indeed, the accelerated recovery of platelet and white cell count, consistent with observations from the previous trial in pancreatic cancer, demonstrates that the marrow microenvironments required for thrombopoiesis, erythropoiesis, and granulopoiesis remain healthy.

Collectively, the results show that ODSH, and other heparin derivatives that likewise inhibit binding of CXCL12 to CXCR4, can be used to mobilize HSCs from the bone marrow, without interfering with the ability of the marrow to be repopulated and support multi-lineage hematopoiesis. The results also show that ODSH, and other heparin derivatives that likewise inhibit binding of CXCL12 to CXCR4, can be used to mobilize other hematopoietic cell types, such as T lymphocytes, from the bone marrow.

### 5.2. Methods of Hematopoietic Stem Cell Transplantation

5.2.1. Methods of Mobilizing Donor HSC Cells

Accordingly, in a first aspect, methods of obtaining hematopoietic stem cells (HSCs) for transplantation are provided. The method comprises administering to a donor subject a heparin derivative capable of inhibiting, reducing, or abrogating binding of CXCL12 to CXCR4, in an amount and for a period effective to mobilize HSC cells from a donor; and obtaining HSC cells from the donor's peripheral blood. The CXCL12-interacting heparinoid is referred to herein as a CXCL12-interacting heparinoid.

“Hematopoietic stem cell” or “HSC” refers to a stem cell that is capable of propagating cells of the lymphoid, myeloid, and erythroid lineages. HSCs are capable of propagating cells which differentiate into erythrocytes (red blood cells), platelets, granulocytes (such as neutrophils, basophils, and eosinophils), macrophages, B-lymphocytes, T-lymphocytes, and Natural Killer (“NK”) cells. HSCs also display self-renewal properties, which is the ability to generate new HSCs.

HSCs can be identified based on pluripotency, as described above. HSCs can also be identified by presence or absence of one or more cell surface markers for HSCs. In some embodiments, a human HSC can be identified based on presence or absence of one or more cell markers selected from, but not limited to, CD34, CD38, CD90, CD105, CD150, CD48, CD16, CD32, Lin, Thy, c-kit (CD117), sca-1, Tie, CD133, CD7, and CD45RA. In some embodiments, the cell markers of human HSC may also be detected using metabolic markers or dyes such as rhodamine 123, Hoechst 33342, Pyronin-Y, and BODIPY-F11-labeled amino-acetaldehyde (BAAA). In some embodiments, human HSCs are characterized by presence of at least the CD34⁺ marker. In certain embodiments, the human HSCs can be identified by one or more of the cell marker profiles shown in Table I.

TABLE I

CD34⁺
CD34⁺ CD38⁻
CD34⁺ Lin⁻ Thy1⁺
CD34⁺ c-kit⁺
CD34⁺ Tie⁺
CD34⁺ CD133⁺
CD34⁻ Lin⁻ CD133⁻ CD7⁻
CD34⁺ CD38⁻ Lin⁻ Rhodamine123^{low}
CD34+ CD38− Lin− CD45RA− Rhodamine1231ow CD49f+
CD34+ CD38− Lin− CD45RA− CD90+

The CXCL12-interacting heparinoid is administered in an amount and for a time effective to mobilize HSC cells from the bone marrow to the peripheral blood and/or peripheral tissue. HSCs are then obtained from the peripheral blood and/or peripheral tissue, for example by leukapheresis.

In various embodiments, the HSCs obtained from the donor are isolated. In certain embodiments, the HSCs obtained from the donor are purified. In some embodiments, the HSCs are isolated and purified.

“Isolated” in the context of HSCs refers to a preparation of HSCs which has been separated from other components and/or cells which naturally accompany the cell in a tissue, blood, or mammal. Isolating HSCs can be accomplished using various standard methods, such as centrifugal, electrical, or size-based methods. An exemplary method of isolating HSCs is apheresis, such as leukapheresis.

In some embodiments, the HSCs are purified. “Purified” in the context of HSCs refers to a preparation of HSCs which has been enriched in HSCs, purified from other cell types with which it is normally associated in its naturally-occurring state. In some embodiments, a purified preparation of HSCs has about 50% or more, about 60% or more, about 70% or more, about 80% or more of the cells being HSCs in the purified preparation. A “substantially purified” preparation of HSCs has more than 80%, about 85% or more, about 90% or more, or 95% or more of the cells being HSCs in the substantially purified preparation.

Various methods can be used to prepare isolated, purified, or substantially purified HSCs for transplantation. In some embodiments, the isolated, purified, or substantially purified HSCs can be prepared by fluorescence activated cell sorting (FACS), affinity chromatography, affinity selection on solid matrixes (e.g., magnetic beads), apheresis, or combinations thereof. In certain embodiments, isolating and/or purifying the HSCs uses an affinity agent, for example antibodies that specifically bind to cell surface markers present on HSCs (Spangrude et al., Science. 241(4861):58-62 (1988); Shizuru et al., Biol Blood Marrow Transplant. 2(1):3-14 (1996); Wang et al., Proc Nat'l Acad Sci USA. 94(26):14632-14636 (1997). In some embodiments, the HSCs can be isolated and/or purified by leukapheresis of collected blood (see, e.g., Cassens et al., Transfusion 44(11):1593-1602 (2004); Schreiner et al., Transfusion 38:1051-1055 (1998)).

In certain embodiments, the HSC are matured during the mobilization, isolation and/or purification procedure such that the HSC have more limited multi-potency than the original pluripotent HSC, and may only be able to differentiate into the myeloid or lymphoid lineages. As a result, in certain embodiments, the isolated or purified HSCs may consist of myeloid or may consist of lymphoid cells.

In some embodiments, the donor subject is the same individual as the ultimate recipient of the cells, i.e., an autologous donor. Autologous transplantation of HSCs is performed, e.g., where a treatment regimen, such as high dose chemotherapy and/or whole body irradiation, is used to treat the underlying disease, thereby destroying or suppressing the hematopoietic system of the subject. Autologous HSCs, which are removed and in some instances stored prior to treatment of the subject with the my elotoxic treatment regimen, are transplanted back into the subject to reconstitute the hematopoietic system.

In certain embodiments, the autologous HSCs are subject to additional treatments prior to transplantation back into the recipient.

In some embodiments, for example, the autologous HSCs obtained from the subject are further treated or purified to remove any disease cells, such as cancer cells, prior to transplantation back into the subject. Such treatments and purification, also referred to as “purging,” of HSC preparations to remove disease cell can include, among others, use of antibodies directed against cell surface markers expressed in disease cells (e.g., anti-CD19, anti-CD20, anti-CD96, etc.; see, e.g., Webb et al., Biol Blood Marrow Transplant. 8(8):429-34 (2002); Staudinger et al., Oncoimmunology 2 (6):e24500 (2013)), viral based purging (Thirukkumaran et al., Bone Marrow Transplant. 40(1):1-12 (2007)), and chemotherapeutic treatments (e.g., Gleevac, etc.).

In some embodiments, the autologous HSCs are obtained following treatment of the subject for the underlying disease, thereby reducing the risk of presence of disease cells in the autologous HSC preparations. Also referred to as “in vivo purging,” the subject can be treated with an antibody therapeutic targeting the disease or treated with chemotherapy that does not destroy or suppress the hematopoietic system prior to obtaining the graft HSCs for transplantation. In certain embodiments, the purged preparation of HSCs can be further purified, such as by FACS or affinity selection (e.g., chromatography), to separate the HSCs from disease cells.

In some embodiments, the donor subject is not the same individual as the ultimate recipient of the cells, i.e., an allogeneic donor. In typical allogeneic embodiments, allogeneic HSC transplantation uses HSCs obtained from donors who are selected based on matching at three or more loci of the human lymphocyte antigen (HLA) complex. Generally, a perfect match at the HLA loci is preferred. Allogeneic donors can be related, such as a closely HLA matched sibling; syngeneic, i.e., a monozygotic or ‘identical’ twin of the subject; or unrelated, i.e., a donor who is not related but who has a close degree of HLA matching.

In some embodiments, the allogeneic HSCs can be further isolated or purified, and/or subject to further manipulation. In some embodiments, the allogeneic HSCs are subject to additional treatments to expand the population of allogeneic HSCs or manipulated by recombinant methods to introduce heterologous genes or additional functionality to the allogeneic HSCs prior to transplantation into the recipient subject. In certain embodiments, the additional treatment leads to maturation of the HSCs.

HSCs obtained from a donor, either autologous or allogeneic, can be subject to additional treatments prior to transplantation into a recipient subject. In some embodiments, the HSCs are treated to expand the population of HSCs, for example by culturing one or more HSCs in a suitable medium (see, e.g., WO 95/05843; WO 95/03693; WO 95/08105; U.S. Pat. No. 8,506,955; incorporated herein by reference). Media useful for expansion of HSCs include, among others, Dulbecco's MEM, IMDM, X-Vivo 15 (serum-depleted, Cambrex), RPMI-1640 and StemSpan (Stem Cell Technologies). In some embodiments, the cell culture medium is serum free (e.g., StemSpan: Stem Cell Technologies). For promoting expansion, the media is supplemented (e.g., conditioned) with growth factors and/or cytokines, including among others, angiopoietin, fibroblast growth factor (FGF) (e.g., FGF-1 or FGF-2), insulin-like growth factor (e.g., IGF-2, or IGF-1), thrombopoietin (TPO), stem cell factor (SCF), and combinations thereof. Concentrations of cytokines and growth factors can range from about 0.1 ng/mL to about 500 ng/mL, from about 1 ng/mL to about 200 ng/mL, from about 10 ng/ml to 100 ng/ml, depending on the type of cytokine and growth factor (see, e.g., U.S. Pat. No. 8,506,955). Other cytokines that can be present include, among others, G-CSF, GM-CSF, IL-1α, IL-11, and combinations thereof. Appropriate concentrations of cytokines can be readily determined by one of ordinary skill in the art.

In some embodiments, the HSCs, either autologous or allogeneic, are manipulated by recombinant methods to introduce heterologous genes, manipulated to correct genetic defects, and/or introduce additional functionality to the HSCs prior to transplantation. In some embodiments, a functioning wild type gene is introduced into the HSC to correct a genetic 32457/33099/DOCS/3922048.3 defect, for example, congenital hematopoietic disorders (e.g., β-thalassemia, Fanconi anemia, hemophilia, sickle cell anemia, etc.); primary immunodeficiencies (e.g., adenosine deaminase deficiency, X-linked severe combined immunodeficiency, chronic granulomatous disease, Wiskott-Aldrich syndrome, Janus kinase 3 deficiency, purine nucleoside phosphorylase (PNP) deficiency, leukocyte adhesion deficiency type 1, etc.); and congenital metabolic diseases (e.g., mucopolysaccharidosis (MPS) types I, II, III, VII, Gaucher disease, X-linked adrenoleukodystrophy, etc.) (see, e.g., Hatada et al., Proc Nat'l Acad Sci USA. 97(25):13807-13811 (2000); Bortug et al., N Engl J Med 363:1918-1927 (2010)). In certain embodiments, the HSCs are subjected to gene manipulation by recombinase systems, such as genome editing using CRISPR/Cas9 system or Cre/Lox recombinases. For example, the recombinase systems can be used to ablate genes or correct gene defects (see, e.g., Mandal et al., Cell Stem Cell. 15(5):643-52 (2014); Meissner et al., Methods Enzymol. 546:273-95 (2014)). In various embodiments, other methods of altering the functionality of HSCs include, among others, introduction of antisense nucleic acids, ribozymes, and RNAi (see, e.g., An et al., Proc Natl Acad Sci USA. 104(32):13110-13115 (2007); Schomber et al., Blood 103(12):4511-4513 (2004)).

In certain embodiments, the method of obtaining HSCs further comprises administering at least a second HSC mobilizing agent adjunctively with the CXCL12-interacting heparinoid. By adjunctive administration is intended that the second HSC mobilizing agent is administered in sufficient temporal proximity to the administration of the CXCL12-interacting heparinoid to enhance HSC mobilization. Enhancement of HSC mobilization includes increase in the number or concentration of HSCs in the peripheral blood and/or tissues, and/or increase in the number or concentration of desired subsets of HSCs, such as detectable by cell marker phenotype, in the peripheral blood and/or tissues.

In some embodiments, the second HSC mobilizing agent is administered prior to administration of the CXCL12-interacting heparinoid. In certain embodiments, the second HSC mobilizing agent is administered concurrently with administration of the CXCL12-interacting heparinoid. In various embodiments, the second HSC mobilizing agent is administered subsequent to treatment with the CXCL12-interacting heparinoid. In some embodiments, the second HSC mobilizing agent is administered before, during, and optionally after the CXCL12-interacting heparinoid is administered.

In some embodiments, the second HSC mobilizing agent is selected from granulocyte-colony stimulating factor (G-CSF), glycosylated G-CSF, pegylated G-CSF, granulocyte macrophage colony stimulating factor (GM-CSF), CXCR4 antagonists (e.g., plerixafor), integrin α4β1 antagonists (e.g., BIO5192), cyclophosphamide, 5-fluorouracil, cisplatin, etoposide, ifosfamide, cytarabine, Me6TREN (CXCR4 antagonist), and combinations thereof (see, e.g., Sheppard et al., Biol Blood Marrow Transplant. 18(8):1191-1203 (2012)).

In some embodiments, the CXCL12-interacting heparinoid is administered to a donor subject who is a poor mobilizer. A “poor mobilizer” refers to a donor subject who has fewer than 20 CD34⁺ cells/μL of peripheral blood, particularly less than 15 CD34⁺ cells/μL of peripheral blood, following mobilization with G-CSF. An exemplary course of G-CSF treatment for mobilization is about 10 mcg/kg/day by subcutaneous administration for multiple days (see, e.g., Neupogen® (filgrastim) Product Label, Amgen Ltd; Granocyte® (lenograstim) Product Label, Chugai Pharma Ltd.). Approximately about 15% to about 20% of donor subjects are typically poor mobilizers.

In the method, the donor is administered the CXCL12-interacting heparinoid in an amount, at a time, and for a period effective to mobilize HSCs, and the HSCs are collected after a time sufficient for HSCs to move to the peripheral blood and/or peripheral tissue.

In certain embodiments, the donor subject is treated with the CXCL12-interacting heparinoid up to the time of isolating the HSCs from the peripheral blood and/or peripheral tissue. In certain embodiments, the donor subject is treated with the CXCL12-interacting heparinoid up to and during the time of HSCs are obtained from the peripheral blood and/or peripheral tissue. In certain embodiments, the donor subject is treated with the CXCL12-interacting heparinoid followed by a period during which no CXCL12-interacting heparinoid is administered prior to obtaining the HSCs. In various embodiments, the heparinoid is administered and the HSCs collected when the concentration of HSCs in the peripheral blood is at or near its maximum or peak level, for example as determined by measuring HSC levels in peripheral blood using a cell marker for HSCs.

In some embodiments, the HSCs are obtained from the peripheral blood and/or peripheral tissue up to 1 hr to about 24 hours after initiating treatment with the CXCL12-interacting heparinoid. In some embodiments, the HSCs are obtained from the peripheral blood and/or peripheral tissue up to 4 hours, 5 hours, 6 hours, 7 hours, 8 hours, 12 hours, 16 hours, 24 hours or more after initiating treatment with the CXCL12-interacting heparinoid. In certain embodiments, the HSCs are obtained from the peripheral blood and/or peripheral tissue up to 2 days, 3 days, 4 days, 5 days, 6 days, 7 days or more after initiating treatment with the CXCL12-interacting heparinoid. In some embodiments, the HSCs are obtained from the peripheral blood and/or peripheral tissue up to 1 week, 2 weeks, 3 weeks, or 4 weeks or more after initiating treatment with the CXCL12-interacting heparinoid. In certain embodiments, the HSCs are obtained from the peripheral blood and/or peripheral tissue up to 1 month, 2 months or 3 months or more after initiating treatment with the heparinoid.

As further described below, various doses of CXCL12-interacting heparinoid can be used. A person of skill in the art can determine the appropriate dose, for example, by measuring the levels of HSCs in the peripheral blood. In some embodiments, the CXCL12-interacting heparinoid is administered at a high dose or a high to moderate dose, as described herein.

In various embodiments, the HSCs obtained as above-described are then administered to an HSC transplant recipient.

5.2.2. Methods of Conditioning an HSC Transplant Recipient

In another aspect, methods are provided for conditioning an HSC transplant recipient to enhance the establishment or engraftment of donor (also termed graft, or transferred) HSCs in the recipient. The method comprises administering a CXCL12-interacting heparinoid in an amount and for a time period sufficient to deplete the recipient's bone marrow of cellular elements, at a time when such depletion enhances the establishment or grafting of donor HSCs in the recipient.

Without being bound by theory, the CXCL12-interacting heparinoid administered in such amount and for such period is believed to mobilize the recipient's bone marrow cells to the periphery, thereby reducing competition by endogenous cells residing in the bone marrow, particularly the recipient's HSCs in the bone marrow, for physiological niches required for engraftment.

The CXCL12-interacting heparinoid is administered in appropriate temporal proximity to administration of the graft HSCs as to enhance engraftment of graft HSCs.

In some embodiments, the recipient subject in need of HSC transplantation is treated with the CXCL12-interacting heparinoid up to, but not at the time of, graft HSC transplantation. In certain embodiments, the recipient subject is treated with the CXCL12-interacting heparinoid up to and during the time of graft HSC transplantation. In certain embodiments, the subject is treated with the CXCL12-interacting heparinoid, followed by a period during which no heparinoid is administered, prior to graft HSC transplantation.

In certain embodiments, the HSCs are transplanted up to 1 hr to about 24 hrs after initiating treatment with the CXCL12-interacting heparinoid. In certain embodiments, the graft HSCs are transplanted up to 4 hours, 5 hours, 6 hours, 7 hours, 8 hours, 12 hours, 16 hours, 24 hours or more after initiating treatment with the CXCL12-interacting heparinoid. In certain embodiments, the graft HSCs are transplanted up to 1 day, 2 days, 3 days, 4 days, 5 days, 6 days, 7 days or more after initiating treatment with the heparinoid. In certain embodiments, the graft HSCs are transplanted up to 1 week, 2 weeks, 3 weeks, 4 weeks or more after initiating treatment with the heparinoid. In some embodiments, the HSCs are transplanted up to 1 month, 2 months or 3 months or more after initiating treatment with the heparinoid.

In some embodiments, the CXCL12-interacting heparinoid is administered in an amount effective to mobilize endogenous HSCs into the peripheral blood of the subject.

In certain embodiments, the subject is administered a high dose of the heparinoid, e.g., to maximize the mobilization of endogenous bone marrow cells, particularly HSCs, and thereby enhance engraftment of graft HSCs. In certain embodiments, the subject is administered a high to moderate dose of the heparinoid. For example, the subject can be treated with a bolus of a high dose of the CXCL12-interacting heparinoid followed by a high to moderate dose by continuous infusion.

In some embodiments, the conditioning regimen further comprises adjunctively administering at least a second HSC cell mobilizing agent in combination with the CXCL12-interacting heparinoid. In some embodiments, the second HSC mobilizing agent is selected from, among others, granulocyte-colony stimulating factor (G-CSF), glycosylated G-CSF, pegylated G-CSF, granulocyte macrophage colony stimulating factor (GM-CSF), CXCR4 antagonists (e.g., plerixafor), CXCR4 inhibitors (e.g., POL6326, BKT140, TG-0054 and NOX-A12), VLA4 antagonists (e.g., anti-VLA4 antibodies such as natalizumab), VCAM-1 inhibitors, CD44 antagonists, integrin α4β1 antagonists (e.g., BIO5192), proteasome inhibitors (e.g., bortezomib), parathormone (PTH), CXCL2 (Groβ), cyclophosphamide, 5-fluorouracil, cisplatin, etoposide, ifosfamide, cytarabine, and combinations thereof.

In some embodiments, the second HSC cell mobilizing agent is an agent that activates a protease, such as, but not limited to, matrix metalloproteinase-9 (MMP-9), membrane-type-1-metalloproteinase (MT-1 MMP), cathepsin G, cathepsin K and neutrophil elastase. In certain embodiments, the second HSC cell mobilizing agent is an agent that inhibits a protease inhibitor. In certain other embodiments, the second HSC cell mobilizing agent activates the dipeptidase CD26. In certain embodiments the second HSC cell mobilizing agent causes degradation of one or more of CXCL12, VCAM-1, fibronectin, or OPN. In some embodiments the second HSC cell mobilizing agent leads to reduced cellular adhesion of HSC. In certain other embodiments, the second HSC cell mobilizing agent increases sphingosine-1-phosphate (SIP) abundance or activity (e.g., SIP agonists) in the peripheral blood and/or decreases SP abundance or activity in the bone marrow. In yet certain other embodiments, the second HSC cell mobilizing agent activates one or more of: m-TOR, reactive oxygen species (ROS), heterodimer HIF-1 (e.g., FG-4497) and vascular endothelial growth factor (VEGF).

In some embodiments, the second HSC mobilizing agent is administered prior to, concurrently with, or subsequent to treatment with the CXCL12-interacting heparinoid. In typical embodiments, the HSC mobilizing agent is administered in sufficient temporal proximity to the administration with the CXCL12-interacting heparinoid to enhance engraftment of graft HSCs. In certain embodiments, the second HSC cell mobilizing agent is selected from mobilizing agents that do not suppress HSC niche supportive macrophage and/or osteoblasts, e.g., plerixafor. In certain embodiments, a non-myelosuppressive HSC mobilizing agent is used, e.g., G-CSF.

In certain embodiments, the subject in need of HSC transplantation is treated additionally with a myeloablative regimen. A “myeloablative” regimen refers to treatment that destroys or has a cytotoxic effect on myeloid cells, for example hematopoietic stem cells of the subject, such that the subject is incapable of hematologic recovery, e.g., reconstituting the immune system. An exemplary myeloablative regimen uses cytotoxic doses of chemotherapy (e.g., cyclophosphamide) combined with total body irradiation.

In some embodiments, the subject in need of HSC transplantation is treated additionally with a reduced intensity myeloablative regimen (see, e.g., Bacigalupo et al., Biol. Blood Marrow Transplant. 15(12):1628-1633 (2009)). A “reduced intensity myeloablative” regimen refers to treatment that causes cytopenia, which may be prolonged, and so require HSC transplantation to reconstitute the subject's hematopoietic system, but with the possibility that autologous recovery would eventually occur, although pancytopenia would be of such duration to cause significant morbidity and mortality. The reduced intensity myeloablative regimen generally does not use total body irradiation and uses a lower dose of the cytotoxic chemotherapeutic as compared to a myeloablative regimen.

In some embodiments, the subject in need of HSC transplantation is treated additionally with a non-myeloablative regimen. A “non-myeloablative,” regimen refers to a treatment that does not have a cytotoxic effect on myeloid cells, for example, hematopoietic stem cells. In some embodiments, a non-myeloablative agent used in the methods described herein has a cytotoxic effect on the circulating mature lymphocytes (e.g., NK cells, and T and B lymphocytes) while sparing the progenitor cells, e.g., hematopoietic stem cells, that are capable of reconstituting the immune system. Generally, non-myeloablative regimens typically cause minimal cytopenia, and little early toxicity, but are immunosuppressive to the extent that they usually result in engraftment of donor HSCs.

Certain of these additional treatment regimens can also cause mobilization of HSCs. In various embodiments, the CXCL12-interacting heparinoid is administered prior to, concurrently with, or subsequent to the reduced intensity or non-myeloablative conditioning regimen. In certain embodiments, the heparinoid is administered subsequent to a non-myeloablative treatment regimen.

In certain embodiments, no myelosuppressive treatments are used, for example, in an autologous HSC transplantation setting where the subject is not being treated with myelosuppressive therapy for an underlying disease and/or for myelosuppression of the subject's immune system.

The effect of the conditioning regimen in promoting or enhancing engraftment can be examined by standard methods. Such methods, as also discussed below, include recovery/restoration of myeloid and/or lymphocytic functions, such as recovery/restoration of white blood cells (e.g., white blood cell count) neutrophils (e.g., neutrophil count), platelets (e.g., platelet count) and reticulocytes (e.g., reticulocyte count or hematocrit); increase in number of donor HSCs (e.g., CD34⁺ cells) in peripheral blood and/or bone marrow; and/or survival rate following HSC transplantation.

In some embodiments, the method further comprises administering donor HSC cells to the conditioned recipient.

5.2.3. Methods of HSC Transplantation

In another aspect, methods are provided for HSC transplantation, in which HSC cells are obtained from a donor, and the donor HSC cells are then administered to a suitable recipient.

In certain embodiments, the HSC cells are obtained according to the methods described in Section 5.2.1 above, and then administered to a suitable transplant recipient. In certain embodiments, HSC cells are administered to a recipient conditioned for HSC transplant according to the methods described in Section 5.2.2 above. In various embodiments, HSC cells obtained according to the methods described in Section 5.2.1 above are administered to a recipient conditioned for HSC transplant according to the methods described in Section 5.2.2 above.

HSCs used for transplantation are also referred to herein as “graft HSCs.”

In certain embodiments, the graft HSCs are autologous; that is, as discussed herein, the HSCs are obtained from the subject receiving the HSC transplantation. In certain autologous transplantation embodiments, the subject has a clinical indication selected from, among others, multiple myeloma, non-Hodgkin's lymphoma, Hodgkin's disease, acute myeloid leukemia, neuroblastoma, amyloidosis neuroblastoma, systemic lupus erythematosus (SLE), systemic sclerosis, germ cell tumors (ovarian cancer), breast cancer, prostate cancer, lung cancer, colon cancer, skin cancer, liver cancer, pancreatic cancer, and sarcoma.

In certain autologous transplantation embodiments, the HSCs are recombinantly engineered ex vivo prior to administration, for example to introduce a heterologous gene or additional functionality to the HSCs. For example, autologous HSCs can be recombinantly engineered to introduce a functioning wild-type gene to correct a genetic defect.

In some embodiments, the graft HSCs are allogeneic; that is, as discussed herein, the HSCs are obtained from a donor who is a different individual from the HSC transplant recipient.

In certain allogeneic transplantation embodiments, the recipient has a clinical indication selected from, among others, acute myeloid leukemia, acute lymphoblastic leukemia, chronic myeloid leukemia, chronic lymphocytic leukemia, myeloproliferative disorders, myelodysplastic syndromes, multiple myeloma, Non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, leukocyte adhesion deficiency, and HIV infection.

In certain allogeneic transplantation embodiments, the HSCs are recombinantly engineered ex vivo prior to administration, for example to introduce a heterologous gene or additional functionality to the HSCs. For example, autologous HSCs can be recombinantly engineered to introduce a functioning wild-type gene to correct a genetic defect.

In some embodiments, the graft HSCs are obtained from umbilical cord blood, and administered to recipients conditioned according to the methods described in Section 5.2.2 above. HSCs from umbilical cord blood can be used for allogeneic HSC transplantation, for example to treat hematological malignancies or bone marrow destruction. Significant advantages include a rapid access to umbilical cord blood, which are stored in cord blood banks, and the ability to accept 1 to 2 HLA mismatches, due to infrequent severe graft-versus-host disease (GVHD) as compared to the matched unrelated donor grafts (Barker et al., Curr Opin Oncol. 14(2):160-4 (2002)). HSCs obtained from umbilical cord blood allow use of allogeneic transplant as a curative option for hematological malignancies where no otherwise suitable match donors are available. In certain embodiments, the umbilical cord blood can be from the subject receiving the HSC transplantation, such as when the umbilical cord blood has been obtained at birth of the subject and stored, and then used at a later time to treat the subject, such as when the subject is a child or adult. Procedures for obtaining umbilical cord blood include draining the blood by gravity from the delivered placenta, and draining the blood by venipuncture into collection bags or syringes.

Transplanting of the HSCs can use standard techniques and procedures used by those of skill in the art.

Typically, a therapeutically effective amount of the HSCs is administered to the subject in need thereof. The effective amount of HSCs can range from as few as several hundred to as many as several million or more. It will be appreciated that the number of HSC to be administered will vary depending on the specifics of the disorder to be treated, including but not limited to size or total volume to be treated, as well as the needs and condition of the subject, among other factors familiar to the medical professional. In some embodiments, from about 10³ to about 10⁸ HSC cells (e.g., CD34⁺ cells) per kg body weight are administered or transplanted into the subject. In some embodiments, from about 10⁴ to about 10⁷ HSC cells, particularly from about 10⁵ to about 10⁷ HSC cells, more particularly from about 10⁶ to about 10⁷ HSC cells (e.g., CD34⁺ cells) per kg body weight are administered or transplanted into the subject. Methods of administering or transplanting HSCs are well known in the art and include, for example, intravenous infusion. In various embodiments, the HSC are administered in a single administration. In some embodiments, the HSC are administered in multiple administrations. Multiple administrations can be provided over periodic time periods such as an initial treatment regime of 3 to 7 consecutive days, and then repeated at other times.

5.2.4. Methods for Enhancing Recovery of the Hematopoietic System after HSC Transplantation

In another aspect, the CXCL12-interacting heparinoid is used to enhance recovery of the hematopoietic system following HSC transplantation.

The method comprises administering CXCL12-interacting heparinoid to an HSC transplant recipient in an amount, at a time, and for a period effective to enhance the recovery of the hematopoietic system. In certain embodiments, the function of at least one hematopoietic lineage is enhanced as compared to historic controls. In typical embodiments, the function is enhanced by the increase in numbers of functioning cells. Thus, in various embodiments, increased numbers of cells in at least one hematopoietic lineage in the bone marrow or peripheral blood are achieved, as compared to historic controls. In certain embodiments, enhanced recovery is measured as increased numbers of platelets in the peripheral blood.

In some embodiments, an amount of a CXCL12-interacting heparinoid effective to enhance recovery of myeloid and/or lymphocytic function of the subject is administered. In various embodiments, administering the CXCL12-interacting heparinoid is sufficient to enhance recovery of one or more of white blood cells (e.g., white blood cell count), neutrophils (e.g., neutrophil count), platelets (e.g., platelet count), reticulocytes (e.g., hematocrit); and CD34⁺ HSCs, particularly an increase in number of donor HSCs (e.g., donor CD34⁺ cells in recipient peripheral blood). Other standard markers known to those of skill in the art can be used in the method. In some embodiments, administering an effective amount of the CXCL12-interacting heparinoid following HSC transplantation shortens the time to recovery/restoration of myeloid and/or lymphocytic function as compared to subjects not treated with the heparinoid, including shortening the time for recovery/restoration of one or more of white blood cells (e.g., white blood cell count), neutrophils (e.g., neutrophil count), platelets (e.g., platelet count), reticulocytes (e.g., reticulocyte count), and CD34⁺ HSCs, particularly an increase in number of donor HSCs (e.g., donor CD34⁺ cells in recipient peripheral blood).

In certain embodiments, platelet count is considered sufficiently restored/recovered if the platelet count is at least 15,000 to 20,000/mm³. In certain embodiments, the platelet count is stable at the specified levels for at least 2 days, 3 days, 4 days, 5 days, 6 days, 7 days or more. In certain embodiments, the days are consecutive days.

In certain embodiments, neutrophil count is sufficiently restored/recovered if the neutrophil count (absolute neutrophil count or ANC) is at least 500 to 1,000/mm³. In certain embodiments, the neutrophil count is stable at the specified levels for at least 2 days, 3 days, 4 days, 5 days, 6 days, 7 days or more. In certain embodiments, the days are consecutive days.

In certain embodiments, reticulocyte count or hematocrit is sufficiently restored/recovered if the hematocrit is at least 25% to 30%. In certain embodiments, the hematocrit is stable at the specified levels for at least 2 days, 3 days, 4 days, 5 days, 6 days, 7 days or more. In certain embodiments, the days are consecutive days.

In certain embodiments, CD34⁺ level (total including donor HSCs) is sufficiently restored/recovered if the CD34⁺ count is about 1.4 CD34⁺ cells/4 to about 2.8 CD34⁺ cells/4 of recipient peripheral blood (see, e.g., Waller et al., Cytotherapy 1(1):21-9 (1999)). In certain embodiments, the CD34⁺ count is stable at the specified levels for at least 2 days, 3 days, 4 days, 5 days, 6 days, 7 days or more. In certain embodiments, the days are consecutive days.

In some embodiments, an effective amount of the CXCL12-interacting heparinoid is administered to increase the probability of survival following HSC transplantation. The method comprises administering to a subject who has received transplanted or graft HSCs an effective amount of a CXCL12-interacting heparinoid to increase the probability of survival of the subject.

In embodiments of the methods, the recipient of the HSC transplantation is treated with the CXCL12-interacting heparinoid in an amount and at a time effective to enhance recovery of the hematopoietic system, e.g., the heparinoid is administered in sufficient temporal proximity to the HSC transplantation to enhance recovery of hematopoietic system of the subject and/or enhance engraftment of transplanted HSCs. In certain embodiments, sufficient time is provided for initial establishment of the transplanted HSCs, e.g., in the bone marrow, prior to administration of the heparinoid. In certain embodiments, the subject is treated with the CXCL12-interacting heparinoid during and after HSC transplantation to enhance recovery of hematopoietic system function.

In certain embodiments, the transplant recipient is treated with the CXCL12-interacting heparinoid up to 1 hr to about 24 hrs. In certain embodiments, the transplant recipient is treated with the heparinoid up to 4 hours, 5 hours, 6 hours, 7 hours, 8 hours, 12 hours, 16 hours, even up to 24 hours. In certain embodiments, the transplant recipient is treated with the heparinoid up to 1 day, 2 days, 3 days, 4 days, 5 days, 6 days, 7 days or more. In certain embodiments, the transplant recipient is treated with the heparinoid up to 1 week, 2 weeks, 3 weeks, 4 weeks or more. In some embodiments, the transplant recipient is treated with the heparinoid up to 1 month, 2 months, 3 months, 4 months, 5 months or 6 months or more, including up to 1 year or 2 years. In various embodiments, the transplant recipient is treated for the period above after transplantation of HSCs. In various embodiments, the transplant recipient is treated with the heparinoid until recovery of hematopoietic system function.

In various embodiments, the subject has been treated by transplantation with autologous HSCs.

In some embodiments, the subject has been treated by transplantation with allogeneic HSCs.

In certain embodiments, the HSCs have been treated to remove diseased cells, such as when autologous HSCs are used to reconstitute a subject's hematopoietic system following myeloablative treatments for an underlying disease.

In certain embodiments, the HSCs have been recombinantly engineered, for example to introduce a heterologous gene or additional functionality to the HSCs.

In some embodiments, the subject has been treated with a myelosuppressive regimen prior to transplantation of HSCs. As described herein, the myelosuppressive regimen can be a myeloablative, a reduce intensity myeloablative, or a non-myeloablative treatment regimen, for example to suppress the subject's immune system to reduce the risk of rejection of transplanted HSCs and/or to treat an underlying disease or disorder.

In the embodiments of the method, the CXCL12-interacting heparinoid is administered in an amount effective to achieve the desired therapeutic effect. In some embodiments, a low dose of the CXCL12-interacting heparinoid is administered to enhance recovery of hematopoietic system and/or enhance engraftment of the transplanted HSCs. In some embodiments, a moderate dose of the CXCL12-interacting heparinoid is administered to enhance recovery of hematopoietic system and/or enhance engraftment of the transplanted HSCs. In some embodiments, a high dose of the CXCL12-interacting heparinoid is administered to enhance recovery of hematopoietic system and/or enhance engraftment of the transplanted HSCs. In some embodiments, a low to moderate dose of the CXCL12-interacting heparinoid is administered. In some embodiments, a moderate to high dose of the CXCL12-interacting heparinoid is administered.

### 5.3. T Cell Immunotherapy Methods

5.3.1. Methods of Mobilizing Donor T Lymphocytes

In another aspect, methods of obtaining T lymphocytes (also called, “T cells”) for transplantation are provided. The method comprises administering to a donor subject a CXCL12-interacting heparinoid, then obtaining T lymphocytes from the donor's peripheral blood.

In preferred embodiments, the method comprises administering a CXCL12-interacting heparinoid in an amount, and for a period effective to increase in the peripheral blood of a donor the concentration of T lymphocytes having at least one desired phenotype; and obtaining T lymphocytes from the donor's peripheral blood.

In typical embodiments, the desired phenotype is defined by surface markers.

In certain embodiments, the T lymphocytes are CD4⁺.

In certain embodiments, the T lymphocytes are CD8⁺.

In certain embodiments, the T lymphocytes are CD45RA⁺, CD62L⁺, CD28⁺, CD95⁻.

In certain embodiments, the T lymphocytes are CD45RA⁺, CD62L⁺, CD28⁺, CD95⁺.

In certain embodiments, the T lymphocytes are CD45RO⁺, CD62L⁺, CD28⁺, CD95⁺.

In certain embodiments, the T lymphocytes are CD45RO⁺, CD62L⁻, CD28⁺.

In certain embodiments, the T lymphocytes are CD45RO⁺, CD62L⁻, CD28⁺, CD95⁺.

In certain embodiments, the T lymphocytes are CD45RO⁺, CD62L⁻, CD28⁺, Perforin^{hi}, Gzmb^{hi}.

In certain embodiments, the T lymphocytes are CD45RO⁺, CD62L⁻, CD28⁺, CD95⁺, Perforin^{hi}, Gzmb^{hi}.

In certain embodiments, the T lymphocytes are naïve T lymphocytes.

In certain embodiments, the T lymphocytes are memory T lymphocytes.

In certain embodiments, the T lymphocytes so obtained are isolated. In various embodiments, the T lymphocytes are purified. In various embodiments, the T lymphocytes are both isolated and purified.

“Isolated” in the context of T lymphocytes, refers to a preparation of T lymphocytes which has been separated from other components and/or cells which naturally accompany the cell in a tissue, blood, or mammal. Isolating T cells can be accomplished using various standard methods, such as centrifugal, electrical, or size-based methods. An exemplary method of isolating T lymphocytes is apheresis, such as leukapheresis.

“Purified” in the context of T lymphocytes refers to a preparation of T lymphocytes which has been enriched in T lymphocytes, i.e., purified from other cell types with which it is normally associated in its naturally-occurring state. In some embodiments, a purified preparation of HSCs has about 50% or more, about 60% or more, about 70% or more, about 80% or more of the cells being T lymphocytes in the purified preparation. A “substantially purified” preparation of T lymphocytes has more than 80%, about 85% or more, about 90% or more, or 95% or more of the cells being T lymphocytes in the substantially purified preparation.

Various methods can be used to prepare isolated, purified, or substantially purified T lymphocytes for immunotherapy. In various embodiments, the isolated, purified, or substantially purified T lymphocytes are prepared by one or more of fluorescence activated cell sorting (FACS), affinity chromatography, affinity selection on solid matrixes (e.g., magnetic beads), apheresis, and combinations thereof. In certain embodiments, isolating and/or purifying the T lymphocytes uses an affinity agent, for example antibodies that specifically bind to cell surface markers present on T lymphocytes.

In certain embodiments, the donor subject is treated with the CXCL12-interacting heparinoid up to the time T lymphocytes are obtained from the peripheral blood and/or peripheral tissue. In certain embodiments, the donor subject is treated with the CXCL12-interacting heparinoid up to and during the time T lymphocytes are obtained from the peripheral blood and/or peripheral tissue. In certain embodiments, the donor subject is treated with the CXCL12-interacting heparinoid followed by a period during which no heparinoid is administered prior to obtaining the T lymphocytes. In various embodiments, the T lymphocytes are obtained when the concentration of T lymphocytes in the peripheral blood is at or near its maximum or peak level. In certain embodiments, the T lymphocytes are obtained when the concentration of T lymphocytes having a desired phenotype are at or near maximum or peak concentration.

In some embodiments, the T lymphocytes are obtained from the peripheral blood and/or peripheral tissue up to 1 hr to about 24 hours after initiating treatment with the CXCL12-interacting heparinoid. In some embodiments, the T lymphocytes are obtained from the peripheral blood and/or peripheral tissue up to 4 hours, 5 hours, 6 hours, 7 hours, 8 hours, 12 hours, 16 hours, 24 hours or more after initiating treatment with the CXCL12-interacting heparinoid. In certain embodiments, the T lymphocytes are obtained from the peripheral blood and/or peripheral tissue up to 2 days, 3 days, 4 days, 5 days, 6 days, 7 days or more after initiating treatment with the CXCL12-interacting heparinoid. In some embodiments, the T lymphocytes are obtained from the peripheral blood and/or peripheral tissue up to 1 week, 2 weeks, 3 weeks, or 4 weeks or more after initiating treatment with the CXCL12-interacting heparinoid. In certain embodiments, the T lymphocytes are obtained from the peripheral blood and/or peripheral tissue up to 1 month, 2 months or 3 months or more after initiating treatment with the CXCL12-interacting heparinoid.

In typical embodiments, the T lymphocytes are then cultured ex vivo.

In certain embodiments, T lymphocytes obtained from the donor, such as tumor infiltrating lymphocytes (“TIL”s), are expanded in culture and selected for antigen specificity without altering their native specificity (Stevanovic et al., J. Clin. Oncol., EPub ahead of print, 10.1200/JCO.2014.58.9093 (2015); Dudley et al., J. Clin. Oncol. 23(10):2346-2357 (2005), incorporated herein by reference in their entireties). In certain embodiments, the T lymphocytes in culture are contacted with at least one of IL-2, interferon alpha, or anti-CD33 antibodies.

In certain embodiments, the T lymphocytes are contacted with an agent that induces checkpoint blockade in the T lymphocytes, wherein the checkpoint blockade inhibits abundance or function of cytokine-inducible SH2-containing protein (CISH) or increases abundance or activity of Phospholipase C-gamma 1 (PLC-gamma) in the T lymphocytes. In other embodiments, the agent induces checkpoint blockade by inhibiting at least one or more of Programmed Cell Death Protein 1 (PCDP1/PD-1/CD279) (e.g., PD-1 antagonists such as Pembrolizumab or Nivolumab) or PD-1 ligand 1 (PDL-1/CD274).

In certain embodiments, T lymphocytes obtained from the donor are engineered ex vivo to express chimeric antigen receptors of predetermined specificity.

In typical CAR embodiments, the T lymphocytes are transduced with viral expression vectors that drive expression of CARs. In various embodiments, the CARs include an extracellular domain, such as the binding domain from a scFv, that confers specificity for a desired antigen; a transmembrane domain; and one or more intracellular domains that trigger T-cell effector functions, such as the intracellular domain from CD3ζ or FcRγ and, optionally, one or more co-stimulatory domains drawn, e.g., from CD28 and/or 4-1BB (Jensen and Riddell, Immunological Reviews 257:127-144 (2014), incorporated herein by reference in its entirety).

In certain embodiments, T lymphocytes obtained from the donor are engineered ex vivo, typically by transduction with viral expression vectors, to express at least a portion of T cell receptors, typically non-naturally occurring TCRs, that confer desired specificity for antigen presented in the context of specific allelic forms of HLA class I or class II proteins (Liddy et al., Nat. Med. 18(6):980-988 (2012), incorporated by reference in its entirety).

In certain embodiments, the method further comprises administering at least a second T cell mobilizing agent adjunctively with the CXCL12-interacting heparinoid. By adjunctive administration is intended that the second T cell mobilizing agent is administered in sufficient temporal proximity to the administration of the CXCL12-interacting heparinoid as to increase in the peripheral blood of a donor the concentration of T lymphocytes. In certain embodiments, the second T cell mobilizing agent is administered in sufficient temporal proximity to the administration of the CXCL12-interacting heparinoid as to increase in the peripheral blood of the donor the concentration of T lymphocytes having at least one desired phenotype. In some embodiments, the second mobilizing agent is administered prior to treatment with the CXCL12-interacting heparinoids. In certain embodiments, the second mobilizing agent is administered concurrently with treatment with the CXCL12-interacting heparinoids. In select embodiments, the second mobilizing agent is administered subsequent to treatment with the CXCL12-interacting heparinoid. In certain embodiments, the second mobilizing agent is administered prior to, concurrently with, and subsequent to administration of the CXCL12-interacting heparinoid.

In some embodiments, the second T cell mobilizing agent is selected from, among others, granulocyte-colony stimulating factor (G-CSF), glycosylated G-CSF, pegylated G-CSF, granulocyte macrophage colony stimulating factor (GM-CSF), CXCR4 antagonists (e.g., plerixafor), CXCR4 inhibitors (e.g., POL6326, BKT140, TG-0054 and NOX-A12), VLA4 antagonists (e.g., anti-VLA4 antibodies such as natalizumab), VCAM-1 inhibitors, CD44 antagonists, integrin α4β1 antagonists (e.g., BIO5192), proteasome inhibitors (e.g., bortezomib), parathormone (PTH), CXCL2 (Groβ), cyclophosphamide, 5-fluorouracil, cisplatin, etoposide, ifosfamide, cytarabine, and combinations thereof (see, e.g., Sheppard et al., Biol Blood Marrow Transplant. 18(8):1191-1203 (2012)).

In some embodiments, the second T cell mobilizing agent is an agent that activates a protease, such as, but not limited to, matrix metalloproteinase-9 (MMP-9), membrane-type-1-metalloproteinase (MT-1 MMP), cathepsin G, cathepsin K and neutrophil elastase. In certain embodiments, the second HSC cell mobilizing agent is an agent that inhibits a protease inhibitor. In certain other embodiments, the second HSC cell mobilizing agent activates the dipeptidase CD26. In certain embodiments the second HSC cell mobilizing agent causes degradation of one or more of CXCL12, VCAM-1, fibronectin, or OPN, leading to reduced cellular adhesion of HSC. In certain other embodiments, the second HSC cell mobilizing agent increases sphingosine-1-phosphate (SIP) abundance or activity (e.g., S1P agonists) in the peripheral blood and/or decreases S1P abundance or activity in the bone marrow. In yet certain other embodiments, the second HSC cell mobilizing agent activates one or more of: m-TOR, reactive oxygen species (ROS), heterodimer HIF-1 (e.g., FG-4497) and vascular endothelial growth factor (VEGF). In typical embodiments, the donor subject is the same individual as the ultimate recipient of the cells, i.e., an autologous donor.

As further described below, various doses of CXCL12-interacting heparinoid can be used. A person of skill in the art can determine the appropriate dose, for example, by measuring the levels of T lymphocytes in the peripheral blood. In some embodiments, the CXCL12-interacting heparinoid is administered at a high dose or a high to moderate dose, as described herein.

In various embodiments, the T lymphocytes obtained as above-described are then administered to a recipient in need of T cell immunotherapy. In typical embodiments, the recipient is the same as the donor.

5.3.2. Methods of Conditioning a T Cell Immunotherapy Recipient

In another aspect, methods are provided for conditioning a T cell immunotherapy recipient subject to enhance the establishment or grafting of donor (also termed graft, or transferred) T cells in the recipient. The method comprises administering a CXCL12-interacting heparinoid to the recipient in an amount and for a period sufficient to deplete the recipient's bone marrow of cellular elements, at a time when depleting the recipient's bone marrow of cellular elements enhances establishment or grafting of donor T lymphocytes in the recipient.

In some embodiments, the subject in need of T cell immunotherapy is treated with the CXCL12-interacting heparinoid up to, but not at the time of, administration of the T cells. In certain embodiments, the subject is treated with the CXCL12-interacting heparinoid up to and during the time of T cell administration. In certain embodiments, the subject is treated with the CXCL12-interacting heparinoid, followed by a period during which no heparinoid is administered, prior to administration of the T cells.

In certain embodiments, the T cells are administered up to 1 hr to about 24 hrs after initiating treatment with the CXCL12-interacting heparinoid. In certain embodiments, the T cells are administered up to 4 hours, 5 hours, 6 hours, 7 hours, 8 hours, 12 hours, 16 hours, 24 hours or more after initiating treatment with the CXCL12-interacting heparinoid. In certain embodiments, the T cells are administered up to 1 day, 2 days, 3 days, 4 days, 5 days, 6 days, 7 days or more after initiating treatment with the CXCL12-interacting heparinoid. In certain embodiments, the T cells are administered up to 1 week, 2 weeks, 3 weeks, 4 weeks or more after initiating treatment with the CXCL12-interacting heparinoid. In some embodiments, the T cells are administered up to 1 month, 2 months or 3 months or more after initiating treatment with the CXCL12-interacting heparinoid.

In certain embodiments, the subject is administered a high dose of the CXCL12-interacting heparinoid. In certain embodiments, the subject is administered a high to moderate dose of the CXCL12-interacting heparinoid. For example, the subject can be treated with a bolus of a high dose of the CXCL12-interacting heparinoid followed by a high to moderate dose by continuous infusion.

In some embodiments, the conditioning regimen further comprises adjunctively administering at least a second mobilizing agent in combination with the CXCL12-interacting heparinoid. In some embodiments, the second mobilizing agent is selected from granulocyte-colony stimulating factor (G-CSF), glycosylated G-CSF, pegylated G-CSF, granulocyte macrophage colony stimulating factor (GM-CSF), CXCR4 antagonists (e.g., plerixafor), integrin α4β1 antagonists (e.g., BIO5192), cyclophosphamide, 5-fluorouracil, cisplatin, etoposide, ifosfamide, cytarabine, and combinations thereof.

In certain embodiments, the subject is treated additionally with an immunosuppressive regimen.

In some embodiments, the method further comprises administering T cells to the conditioned recipient.

### 5.4. Administration of CXCL12-Interacting Heparinoid

The methods described herein comprise administering a CXCL12-interacting heparinoid to a subject. The subject is typically human.

5.4.1. Effective Heparin Derivatives

In the methods described herein, the heparin derivative is one capable of inhibiting, reducing, abrogating, or otherwise interfering with the binding of CXCL12 to CXCR4. For convenience, such heparin derivatives are collectively referred to herein as “CXCL12-interacting heparinoids”.

In some embodiments, the CXCL12-interacting heparinoid inhibits binding of CXCL12 to CXCR4 with an IC₅₀ of about 0.05 μg/ml or less, about 0.04 μg/ml or less, about 0.03 μg/ml or less, about 0.02 μg/ml or less, or about 0.01 μg/ml or less in the assay set forth in Example 3. In some embodiments, the CXCL12-interacting heparinoid inhibits binding of CXCL12 to CXCR4 with an IC₉₀ of about 0.7 μg/ml or less, about 0.6 μg/ml or less, about 0.5 μg/ml or less, or about 0.4 μg/ml or less in the assay set forth in Example 3. In some embodiments, the heparinoid is characterized by an IC₅₀ of about 0.01 μg/ml and an IC₉₀ of about 0.5 μg/ml as determined by the method in Example 3. In some embodiments, the CXCL12-interacting heparinoid is capable of inhibiting CXLC12/CXCR4 interaction, as measured by the method set forth in Example 3, which is about the same as an equivalent weight of unfractionated heparin.

In typical embodiments, the CXCL12-interacting heparinoid is capable of effecting at least 20% inhibition of the binding of CXCL12 to CXCR4 in the assay set forth in Example 3 at a concentration that, if achieved in plasma, would not effect substantial anticoagulation. In various embodiments, the CXCL12-interacting heparinoid is capable of effecting at least 25% inhibition of the binding of CXCL12 to CXCR4 in the assay set forth in Example 3 at a concentration that, if achieved in plasma, would not effect substantial anticoagulation. In certain embodiments, the CXCL12-interacting heparinoid is capable of effecting at least 30%, at least 35%, at least 40%, at least 45%, at least 50%, at least 55%, at least 60% inhibition of the binding of CXCL12 to CXCR4 in the assay set forth in Example 3 at a concentration that, if achieved in plasma, would not effect substantial anticoagulation. In specific embodiments, the CXCL12-interacting heparinoid is capable of effecting at least 65%, at least 70%, at least 80%, at least 85%, even at least 90%, 91%, 92%, 93%, 94%, 95% inhibition of the binding of CXCL12 to CXCR4 in the assay set forth in Example 3 at a concentration that, if achieved in plasma, would not effect substantial anticoagulation. In particular embodiments, the CXCL12-interacting heparinoid is capable of effecting at least 96%, 97% even at least 98% inhibition of the binding of CXCL12 to CXCR4 in the assay set forth in Example 3 at a concentration that, if achieved in plasma, would not effect substantial anticoagulation.

In various embodiments, the CXCL12-interacting heparinoid is capable of binding to CXCL12 under physiological conditions.

In preferred embodiments, the CXCL12-interacting heparinoid is a derivative of USP heparin (also known as “unfractionated heparin” or “UFH”) that is substantially desulfated at the 2-O position of α-L-iduronic acid (referred to herein as the “2-O position”) and/or 3-O position of D-glucosamine-N-sulfate (6-sulfate) (referred to herein as the “3-O position”). In preferred embodiments, the 2-O, 3-O-desulfated heparin derivative is not substantially desulfated at the 6-O or N positions.

For purposes of the present disclosure, the percentage desulfation at the 2-O position of a sample of 2-O, 3-O-desulfated heparin derivative (“ODSH”) is defined as the percentage reduction in sulfate functional groups on the 2-O position of the 2-O-sulfo-α-L-iduronic acid residues as compared to the sulfate functional groups on the 2-O positions of the 2-O-sulfo-α-L-iduronic acid residues in a sample of the 6th International Standard for Unfractionated Heparin, NIBSC code 07/328 (“NIBSC standard”). For purposes of the present disclosure, the percentage desulfation at the 3-O position of a sample of ODSH is defined as the percentage reduction in sulfate functional groups on the 3-O position of the 2-deoxy-2-sulfamido-3-O-sulfo-α-D-glucopyranosyl-6-O-sulfate residues as compared to the sulfate functional groups on the 3-O positions of the 2-deoxy-2-sulfamido-3-O-sulfo-α-D-glucopyranosyl-6-O-sulfate residues in a sample of the NIBSC standard.

In some embodiments, the CXCL12-interacting heparinoid is at least 85%, at least 90%, at least 95%, or at least 99% desulfated at the 2-O position. In some embodiments, the CXCL12-interacting heparinoids are at least 85%, at least 90%, at least 95%, or at least 99% desulfated at the 3-O position. In some embodiments, the CXCL12-interacting heparinoids are at least 85%, at least 90%, at least 95%, at least 99% desulfated at the 2-O position and the 3-O position.

For purposes herein, average molecular weight of heparinoids is weight-average molecular weight, Mw, and is determined by size exclusion chromatography according to the USP monograph for Enoxaparin sodium, with USP Heparin MW Calibrant used as an additional calibrant.

In some embodiments, the CXCL12-interacting heparinoids have an average molecular weight from about 2 kDa to about 15 kDa. In some embodiments, the CXCL12-interacting heparinoids have an average molecular weight of at least about 2 kDa, at least about 3 kDa, at least about 4 kDa, at least about 5 kDa, at least about 6 kDa, or at least about 7 kDa. In some embodiments, the CXCL12-interacting heparinoids have an average molecular weight of less than about 15 kDa, less than about 14 kDa, less than about 13 kDa, less than about 12 kDa, less than about 11 kDa, less than about 10 kDa, or less than about 9 kDa. In some embodiments, the average molecular weight of the CXCL12-interacting heparinoid is selected from about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, or a range that includes any of these values as endpoints.

In some embodiments, the substantially 2-O, 3-O desulfated CXCL12-interacting heparinoid for use in the methods described herein are compositions in which the average molecular weight is at least about 8 kDa. In some embodiments, the substantially 2-O, 3-O desulfated CXCL12-interacting heparinoids have an average molecular weight of greater than about 8 kDa. In various embodiments, the substantially 2-O, 3-O desulfated CXCL12-interacting heparinoids have an average molecular weight ranging from about 8 kDa to about 15 kDa. In some embodiments, the substantially 2-O, 3-O desulfated CXCL12-interacting heparinoids for use in the methods described herein have an average molecular weight that ranges in size from about 11 kDa to about 13 kDa.

An exemplary CXCL12-interacting heparinoid is substantially 2-O, 3-O desulfated heparin, referred to herein as ODSH. ODSH for use in the above-described methods can be prepared from bovine or porcine heparin. In an exemplary method of preparing ODSH from porcine heparin, ODSH is synthesized by cold alkaline hydrolysis of USP porcine intestinal heparin, which removes the 2-O and 3-O sulfates, leaving N- and 6-O sulfates on D-glucosamine sugars and carboxylates on α-L-iduronic acid sugars substantially intact (Fryer et al., J. Pharmacol. Exp. Ther. 282: 208-219 (1997), incorporated herein by reference in its entirety). Using this method, ODSH can be produced with an average molecular weight of about 11.7±0.3 kDa. Additional methods for the preparation of substantially 2-O, 3-O desulfated CXCL12-interacting heparinoids may also be found, for example, in U.S. Pat. Nos. 5,668,118, 5,912,237, and 6,489,311, and WO 2009/015183, the contents of which are incorporated herein in their entirety, and in U.S. Pat. Nos. 5,296,471; 5,969,100; and 5,808,021.

In contrast to unfractionated heparin, ODSH is substantially non-anticoagulating: administered to a subject at a dose that is equivalent in weight to a fully-anticoagulating dose of unfractionated heparin, the clotting time measured in an aPTT assay is no greater than 45 seconds, and typically in the upper range of normal, where normal clotting time ranges from about 27 to 35 seconds. By comparison, unfractionated heparin administered to a subject at a fully anticoagulant dose causes time to clot to range from about 60 to about 85 seconds in an aPTT assay.

Thus, in certain preferred embodiments, the CXCL12-interacting heparinoid is substantially non-anticoagulating. In preferred embodiments, the CXCL12-interacting heparinoid, if administered to a subject at a dose that is weight equivalent to a fully-anticoagulating dose of unfractionated heparin, the clotting time measured in an aPTT assay is no greater than 45 seconds.

Another measure of ODSH's anticoagulant activity is its anti-Xₐ activity which can be determined in an assay carried out using plasma treated with Russell viper venom. In specific examples, ODSH exhibited less than 9 U of anticoagulant activity/mg in the USP anticoagulant assay (e.g., 7±0.3 U), less than 5 U of anti-Xₐ activity/mg (e.g., 1.9±0.1 U/mg) and less than 2 U of anti-IIₐ activity/mg (e.g., 1.2±0.1 U/mg) (compared to unfractionated heparin which has an activity of 165-190 U/mg in all three assays; Rao et al., Am. J. Physiol. 299:C97-C110 (2010), incorporated herein by reference in its entirety). Thus, in certain embodiments, the CXCL12-interacting heparinoid exhibits less than 9 U of anticoagulant activity/mg in the USP anticoagulant assay, and/or less than 5 U of anti-Xₐ activity/mg, and/or less than 2 U of anti-IL activity/mg.

Furthermore, ODSH has a low affinity for anti-thrombin III (Kd˜339 μM or 4 mg/ml vs. 1.56 μM or 22 μg/ml for unfractionated heparin), consistent with the observed low level of anticoagulant activity, measured as described in Rao et al., supra, at page C98. Thus, in certain embodiments, the CXCL12-interacting heparinoid has a low affinity for anti-thrombin III (Kd˜339 μM or 4 mg/ml).

In some embodiments, the CXCL12-interacting heparinoids have no more than 40% of the anticoagulating activity of an equal weight of unfractionated heparin by any one or more of the above-described tests. In some embodiments, the CXCL12-interacting heparinoid has no more than 35%, no more than 30%, no more than 20%, no more than 10%, no more than 9%, no more than 8%, no more than 7%, no more than 6%, no more than 5%, no more than 4%, no more than 3%, no more than 2%, or no more than 1% of the anticoagulating activity of an equal weight of unfractionated heparin by any one or more of the above-described tests.

In some embodiments, the CXCL12-interacting heparinoid does not trigger platelet activation and does not induce heparin-induced thrombocytopenia (HIT). Platelet activation can be determined using a serotonin release assay, for example as described in U.S. Pat. No. 7,468,358 and Sheridan et al., Blood 67:27-30 (1986), incorporated herein by reference. In some embodiments, the heparinoid is capable of binding platelet factor 4, also referred to as chemokine (C-X-C motif) ligand 4 (CXCL4).

In some embodiments, the CXCL12-interacting heparinoid is a low molecular weight heparin (LMWH). “Low molecular weight heparin” or “LMWH” refers to heparin fragments that have a mean molecular weight of about 4 to about 6 kDa. In some embodiments, the LMWHs have a molecular weight distribution of about 1000 to about 10000. LMWHs are typically made by chemical or enzymatic depolymerization of heparin, generally unfractionated heparin, and can be further purified to select the appropriate size of the LMWH. The LMWH can be prepared using a number of different separation or fractionation techniques known to and used by those of skill in the art, including, for example, gel permeation chromatography (GPC), high-performance liquid chromatography (HPLC), ultrafiltration, size exclusion chromatography, and the like.

In certain embodiments, the LMWH is selected from the group consisting of bemiparin, nadroparin, reviparin, enoxaparin, parnaparin, certoparin, dalteparin, tinzaparin, and necuparanib.

In typical embodiments, the CXCL12-interacting heparinoid displays bone marrow cell mobilizing activity, particularly HSC mobilizing activity. Where the bone marrow cell mobilizing activity is based on HSC mobilizing activity, various markers indicative of HSCs can be used for detecting mobilization. Exemplary HSC markers are given in Table 1 above.

In some embodiments, the CXCL12-interacting heparinoid is characterized by about 50% or more, 60% or more, 70% or more, 80% or more, 90% or more, or 95% or more of the HSC mobilizing activity of an equivalent weight of unfractionated heparin.

5.4.2. Modes and Routes of Administration

The CXCL12-interacting heparinoid can be administered in the methods by any one or more of a variety of routes.

In certain embodiments, the CXCL12-interacting heparinoid is administered intravenously. In certain intravenous embodiments, the CXCL12-interacting heparinoid is administered by bolus intravenous administration. In some embodiments, a bolus dose is administered over less than a minute, about a minute, about 2 minutes, about 3 minutes, about 4 minutes, or about 5 minutes. In some intravenous embodiments, the CXCL12-interacting heparinoid is administered by continuous intravenous infusion. In other embodiments, the CXCL12-interacting heparinoid is administered by subcutaneous injection. In some embodiments, the CXCL12-interacting heparinoid is administered as one or more bolus intravenous injections preceded and/or followed by continuous intravenous infusion.

5.4.3. Effective Amounts

The CXCL12-interacting heparinoid is administered in amounts effective to achieve the results respectively desired for the methods described above.

In the methods of mobilizing donor HSC cells described above, in typical embodiments the CXCL12-interacting heparinoid is administered in an amount effective to mobilize HSC cells in a donor. In certain embodiments, the CXCL12-interacting heparinoid is administered in an amount effective to mobilize HSCs from the bone marrow to the peripheral blood and/or peripheral tissue of the donor.

In the methods of conditioning a HSC transplant recipient described above, in typical embodiments the CXCL12-interacting heparinoid is administered in an amount effective to deplete the recipient's bone marrow of cellular elements.

In typical embodiments of the methods for enhancing recovery of the hematopoietic system after HSC transplantation described above, the CXCL12-interacting heparinoid is administered in an amount effective to enhance the recovery of the hematopoietic system. In certain embodiments, the amount is effective to enhance the function of at least one hematopoietic lineage. In certain embodiments, the amount is effective to increase the numbers or concentration in peripheral blood of cells of at least one hematopoietic lineage.

In the methods of mobilizing donor T lymphocytes described above, typically the CXCL12-interacting heparinoid is administered in an amount effective to increase in the peripheral blood of a donor the concentration of T lymphocytes having at least one desired phenotype.

In the methods conditioning a T cell immunotherapy recipient, in typical embodiments the CXCL12-interacting heparinoid is administered in an amount sufficient to deplete the recipient's bone marrow of cellular elements.

In some embodiments, the CXCL12-interacting heparinoid is administered as an intravenous bolus. In certain embodiments, the CXCL12-interacting heparinoid is administered in an intravenous bolus of no less than about 1 mg/kg patient body weight. In typical intravenous bolus dosing embodiments, the CXCL12-interating heparinoid is administered at a dose of no more than about 25 mg/kg. In various embodiments, the CXCL12-interacting heparinoid is administered at an intravenous bolus dose of at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, even at least about 10 mg/kg. In some embodiments, the bolus is at least about 15 mg/kg, even at least about 20 mg/kg. In certain preferred embodiments, the bolus is 4 mg/kg. In certain other preferred embodiments, the bolus is 8 mg/kg. In certain preferred embodiments, the bolus is about 20 mg/kg.

In some embodiments, the CXCL12-interacting heparinoid is administered in a bolus of from about 2 to about 25 mg/kg, from about 2 mg/kg to about 20 mg/kg, from about 2 mg/kg to about 15 mg/kg, from about 3 mg/kg to about 10 mg/kg, or from about 4 mg/kg to about 8 mg/kg.

In some embodiments, the CXCL12-interacting heparinoid is administered as an intravenous infusion. In certain embodiments, the infusion is at a dose rate of at least about 0.1 mg/kg/hr, at least about 0.2 mg/kg/hr, at least about 0.3 mg/kg/hr, at least about 0.4 mg/kg/hr, at least about 0.5 mg/kg/hr, at least about 1 mg/kg/hr, even at least about 2 mg/kg/hr. In various embodiments, the CXCL12-interacting heparinoid is administered at an infusion rate of no more than about 5 mg/kg/hr. In certain embodiments, the CXCL12-interacting heparinoid is administered at an infusion rate of no more than about 4 mg/kg/hr, 3 mg/kg/hr, 2 mg/kg/hr, even no more than about 1 mg/kg/hr.

In certain embodiments, the CXCL12-interacting heparinoid is administered by intravenous infusion at a dose rate of about 0.25 mg/kg/hr. In some embodiments, the CXCL12-interacting heparinoid is administered at a dose rate of about 0.375 mg/kg/hr.

In various embodiments, infusions at the above-described dose rates are administered continuously for up to 7 days. In certain embodiments infusions at the above-described dose rates are administered continuously for up to 6 days, 5 days, 4 days, or 3 days. In some embodiments, infusions at the above-described dose rates are administered continuously for up to 2 days or up to 24 hours. In some embodiments, infusions at the above-described rates are administered for up to 4 hours, 5 hours, 6 hours, 7 hours, 8 hours, 12 hours, 16 hours, or up to 24 hours or more. In certain embodiments, the infusions at the above described dose rates are administered for the duration of each cycle of treatment.

In some embodiments, the CXCL12-interacting heparinoid is administered as an initial bolus of about 20 mg/kg, optionally followed by an infusion of up to about 2 mg/kg/hour for at least about 4 hours, up to about 8 hours, up to about 12 hrs, up to about 16 hours, even up to about 24 hours. In one embodiment, the CXCL12-interacting heparinoid is administered as an initial bolus of about 8 mg/kg, optionally followed by an infusion of about 0.5 mg/kg/hour for at least about 8 hours. In some embodiments, the CXCL12-interacting heparinoid is administered as an intravenous bolus at a dose of about 4 mg/kg, optionally followed by an intravenous infusion of the CXCL12-interacting heparinoid at a dose of about 0.25 mg/kg/hr-about 0.375 mg/kg/hr for at least 24 hours. In some embodiments, the CXCL12-interacting heparinoid is administered as an intravenous bolus at a dose of about 4 mg/kg, followed by a continuous intravenous infusion at a rate of 0.25 mg/kg/hr for a total of 7 days.

In the method of mobilizing HSCs to the peripheral blood and/or peripheral tissues, the infusion times can be selected based, in part, on the dose rate and on the time of collection of HSCs after initiating treatment with heparinoid. Similarly, in the method of conditioning a subject for enhancing engraftment of graft HSC cells, the infusion times can be selected based, in part, on the dose rate and on the time for transplantation of graft HSCs into the subject.

For subcutaneous administration, CXCL12-interacting heparinoid can be administered at doses ranging from about 25 mg to about 400 mg, about 50 mg to about 300 mg, or about 75 mg to about 200 mg, in volumes of 2.0 mL or less per injection.

5.4.4. Duration, Frequency, and Adjunctive Administration

The duration and frequency of administering the CXCL12-interacting heparinoid can take into account, among others, the effectiveness of the dosing regimen and the particular method being applied, e.g., mobilization of HSC cells, enhancing engraftment of graft HSC cells; enhancing recovery of hematopoietic system function; mobilizing T lymphocytes; or enhancing engraftment or persistence of T lymphocytes.

In various embodiments, the CXCL12-interacting heparinoid is administered over a period of up to 1 hour. In various embodiments, the CXCL12-interacting heparinoid is administered over a period of up to 4 hours, 5 hours, 6 hours, 7 hours, 8 hours, 12 hours, 16 hours, or up to 24 hours or more. In certain embodiments, the CXCL12-interacting heparinoid is administered over a period of 2 days, 3 days, 4 days, 5 days, 6 days, 7 days or more. In certain embodiments, the CXCL12-interacting heparinoid is administered over a period of up to a week, 2 weeks, 3 weeks, 4 weeks or more. In certain embodiments, the CXCL12-interacting heparinoid is administered over a period of up to 1 month, 2 months, 3 months, 4 months, 5 months, or 6 months or more, including up to 1 year or 2 years.

In various embodiments, CXCL12-interacting heparinoid administration is repeated. For example, in certain embodiments, heparinoid is administered once daily, twice daily, three times daily, four times daily, five times daily, every two days, every three days, every five days, once a week, once every two weeks, once a month, or every other month. In some embodiments, the CXCL12-interacting heparinoid is administered at regular intervals over a period of several days or weeks, followed by a period of rest, during which no heparinoid is administered. For example, in some embodiments, CXCL12-interacting heparinoid is administered for one, two, three, or more days, followed by one, two, three, or more days without heparinoid administration. In another exemplary embodiment, the CXCL12-interacting heparinoid is administered for one, two, three, or more weeks, followed by one, two, three, or more weeks without heparinoid administration. The repeated administration can be at the same dose or at a different dose. The CXCL12-interacting heparinoid can be administered in one or more bolus injections, one or more infusions, or one or more bolus injections followed and/or preceded by infusion.

The frequency of dosing can be based on and adjusted for the pharmacokinetic parameters of the CXCL12-interacting heparinoid, the route of administration, and the desired physiological and/or therapeutic effect. Dosages are adjusted to provide sufficient levels of the CXCL12-interacting heparinoid or to maintain the desired physiological effect and/or a therapeutic effect. Any effective administration regimen regulating the timing and sequence of doses may be used, as discussed herein.

Accordingly, the pharmaceutical compositions can be administered in a single dose, multiple discrete doses, continuous infusion, sustained release depots, or combinations thereof, as required to maintain desired minimum level of the agent. Daily dosages may vary, depending on the specific activity of the particular heparinoid. Depending on the route of administration, a suitable dose may be calculated according to, among others, body weight, body surface area, or organ size. The final dosage regimen will be determined by the attending physician in view of good medical practice, considering various factors that modify the action of drugs, e.g., the agent's specific activity, the responsiveness of the patient, the age, condition, body weight, sex, and the like. Additional factors that may be taken into account include time and frequency of administration, drug combination(s), reaction sensitivities, and tolerance/response to therapy. Further refinement of the dosage appropriate for method involving any of the formulations mentioned herein is done by the skilled practitioner, especially in light of the dosage information and assays disclosed, as well as the pharmacokinetic data observed in clinical trials. The amount and/or frequency of the dosage can be altered, increased, or reduced, depending on the subject's response and in accordance with standard clinical practice. The proper dosage and treatment regimen can be established by monitoring the progress of therapy using conventional techniques known to skilled artisans. Appropriate dosages may be ascertained through use of established assays for determining concentration of the CXCL12-interacting heparinoid in a body fluid or other sample together with dose response data.

In view of the foregoing, in some embodiments of the method of mobilizing HSCs and some embodiments of the method of mobilizing T lymphocytes, the donor subject is treated with the CXCL12-interacting heparinoid over a period of up to 1 hour, 4 hours, 6 hours, 8 hours, 12 hours, 18 hours, even up to 24 hours. In certain embodiments, the donor subject is treated with the CXCL12-interacting heparinoid over a period of up to 2 days, 3 days, 4 days, 5 days, 6 days, 7 days or more. In certain embodiments, the donor subject is treated with the CXCL12-interacting heparinoid over a period of up to 1 week, 2 weeks, 3 weeks, 4 weeks or more. In certain embodiments, the donor subject is treated with the CXCL12-interacting heparinoid over a period of up to 1 month, 2 months, 3 months or more. In certain embodiments, the donor subject is treated for the periods described above up to the time of collecting HSCs from the peripheral blood and/or peripheral tissues. In certain embodiments, the donor subject is treated for the periods described above up to the time of collecting HSCs, and the treatment continued during collecting of HSCs. In some embodiments, the donor subject is treated with the CXCL12-interacting heparinoid for the periods described above followed by a period in which no heparinoid is administered prior to collecting of HSCs from the peripheral blood and/or peripheral tissues.

In some embodiments of the method of enhancing engraftment of HSCs in a subject receiving graft HSC transplantation or enhancing engraftment or persistence of T lymphocytes in a subject receiving T cell immunotherapy, the subject is treated with the CXCL12-interacting heparinoid over a period of up to 1 hour, 4 hours, 6 hours, 8 hours, 12 hours, 18 hours, or even 24 hours. In certain embodiments, the subject is treated with the CXCL12-interacting heparinoid over a period of up to 2 days, 3 days, 4 days, 5 days, 6 days, 7 days or more. In certain embodiments, the subject is treated with the CXCL12-interacting heparinoid over a period of up to 1 week, 2 weeks, 3 weeks, 4 weeks or more. In certain embodiments, the subject is treated with the CXCL12-interacting heparinoid over a period of up to 1 month, 2 months, 3 months or more. In certain embodiments, the subject is treated for the periods described above up to the time of transplanting of graft HSCs or T lymphocytes into the subject. In certain embodiments, the subject is treated for the periods described above up to the time of transplanting of graft HSCs or T lymphocytes, and the treatment continued during transplantation of graft HSCs or T lymphocytes. In some embodiments, the subject is treated with the CXCL12-interacting heparinoid for the periods described above followed by a period in which no heparinoid treatment is given prior to transplanting of graft cells into the subject.

In some embodiments of the method of enhancing engraftment in a subject receiving graft HSCs or graft T lymphocytes, the period in which no heparinoid treatment is given prior to transplanting the transferred cells is about 1 hour to about 24 hours or more, including up to 4 hours, 6 hours, 8 hours, 12 hours, 18 hours, or up to 24 hours from the end of treatment with the CXCL12-interacting heparinoid to the transplanting of graft cells. In certain embodiments, the period from the end of treatment with the CXCL12-interacting heparinoid to the transplanting of graft HSC cells or graft T lymphocytes is up to 1 day, 2 days, 3 days, 4 days, 5 days, 6 days or 7 days or more. In certain embodiments, the period from the end of the treatment with the CXCL12-interacting heparinoid to the transplanting of graft cells is up to 1 week, 2 weeks, 3 weeks or 4 weeks or more. In certain embodiments, the period from the end of the treatment with the CXCL12-interacting heparinoid to the transplanting of graft HSC or T cells is up to 1 month, 2 months, or 3 months or more.

In some embodiments of the method of enhancing recovery of hematopoietic system function in a subject who has received graft HSC transplantation, the subject is treated with the CXCL12-interacting heparinoid over a period of up to 1 hour, 4 hours, 6 hours, 8 hours, 12 hours, 18 hours, or even 24 hours. In certain embodiments, the subject is treated with the CXCL12-interacting heparinoid over a period of up to 1 day, 2 days, 3 days, 4 days, 5 days, 6 days, 7 days or more. In certain embodiments, the subject is treated with the CXCL12-interacting heparinoid over a period of up to a week, 2 weeks, 3 weeks, 4 weeks or more. In certain embodiments, the subject is treated with the CXCL12-interacting heparinoid over a period of up to 1 month, 2 months, 3 months, 4 months, 5 months, 6 months or more, including treatments up to 1 year or 2 years. In various embodiments, the subject is treated with the CXCL12-interacting heparinoid until the recovery of hematopoietic system function. In certain embodiments, the treatment with the CXCL12-interacting heparinoid is initiated subsequent to graft HSC transplantation. In certain embodiments, treatment with the CXCL12-interacting heparinoid is initiated during graft HSC transplantation.

In certain embodiments of the method of enhancing recovery of hematopoietic system function, the treatment with the CXCL12-interacting heparinoid is initiated up to 1 hr to about 24 hrs after transplantation of graft HSCs. In certain embodiments, treatment with the CXCL12-interacting heparinoid is initiated up to 4 hours, 5 hours, 6 hours, 7 hours, 8 hours, 12 hours, 16 hours, 24 hours or more after transplantation of graft HSCs. In certain embodiments, treatment with the CXCL12-interacting heparinoid is initiated up to 1 day, 2 days, 3 days, 4 days, 5 days, 6 days, 7 days or more after transplantation of graft HSCs. In certain embodiments, treatment with the CXCL12-interacting heparinoid is initiated up to 1 week, 2 weeks, 3 weeks, 4 weeks or more after transplantation of graft HSCs. In some embodiments, treatment with the CXCL12-interacting heparinoid is initiated up to 1 month, 2 months or 3 months or more after transplantation of graft HSCs.

In embodiments in which CXCL12-interacting heparinoid is administered to a subject in combination with other therapeutic agents, the CXCL12-interacting heparinoid is administered in a physiologically and/or therapeutically effective temporal proximity to the treatment regimen with the other therapeutic. Administration of a CXCL12-interacting heparinoid can be concurrent with (at the same time), sequential to (at a different time but on the same day, e.g., during the same patient visit), or separate from (on a different day) the treatment with the other therapeutic. In some embodiments, the CXCL12-interacting heparinoid is administered concurrently, sequentially, and/or separately from the other agent or therapy being administered. When administered sequentially or separately, the CXCL12-interacting heparinoid can be administered before, after, or both before and after the other treatment.

In embodiments in which the CXCL12-interacting heparinoid is administered in combination with treatment with another therapeutic agent, the CXCL12-interacting heparinoid can be administrated via the same or different route as the other therapeutic administered in temporal proximity. In some embodiments, the CXCL12-interacting heparinoid is administered concurrently or sequentially by the same route. For example, in some embodiments, the CXCL12-interacting heparinoid and the other therapeutic are administered intravenously, either concurrently or sequentially. Optionally, as part of a treatment regimen, the CXCL12-interacting heparinoid can further be administered separately (on a different day) from the other therapeutic by a different route, e.g., subcutaneously. In some embodiments, the CXCL12-interacting heparinoid is administered intravenously on the same day, either at the same time (concurrently), a different time (sequentially), or both concurrently and sequentially with the other therapeutic, and is also administered subcutaneously on one or more days when the patient is not receiving other treatment. In some embodiments, the CXCL12-interacting heparinoid is administered concurrently or sequentially by a different route. Optionally, as part of a treatment regimen, the CXCL12-interacting heparinoid can further be administered separately (on a different day) from the other therapeutic by the same or different route as that by which the other therapeutic is administered.

Other methods for delivering CXCL12-interacting heparinoids in the methods presented herein can be adapted from those are described in U.S. Pat. No. 4,654,327, which describes oral administration of heparin in the form of a complex with a quaternary ammonium ion; U.S. Pat. No. 4,656,161, which describes a method for increasing the enteral absorbability of heparinoids by orally administering the drug along with a non-ionic surfactant such as polyoxyethylene-20 cetyl ether, polyoxyethylene-20 stearate, other polyoxyethylene (polyethylene glycol)-based surfactants, polyoxypropylene-1 5 stearyl ether, sucrose palmitate stearate, or octyl-beta-D-glucopyranoside; U.S. Pat. No. 4,703,042, which describes oral administration of a salt of polyanionic heparinic acid and a polycationic species; and U.S. Pat. No. 5,714,477, which describes a method for improving the bioavailability of heparinoids by administering in combination with one or several glycerol esters of fatty acids.

### 5.5. Pharmaceutical Compositions and Unit Dosage Forms

In the methods described herein, the CXCL12-interacting heparinoid is administered in the form of a pharmaceutical composition.

In typical embodiments, the pharmaceutical composition comprises the CXCL12-interacting heparinoid and a pharmaceutically acceptable carrier, excipient, and/or diluent, and is formulated for parenteral administration.

5.5.1. Pharmaceutical Compositions Formulated for I.V. Administration

In certain embodiments, pharmaceutical compositions of the CXCL12-interacting heparinoid are formulated in volumes and concentrations suitable for intravenous administration. In some embodiments, the composition is formulated for bolus administration. In certain embodiments, pharmaceutical compositions of the CXCL12-interacting heparinoid are formulated in volumes and concentrations suitable for intravenous infusion.

Typical embodiments formulated for intravenous administration comprise the CXCL12-interacting heparinoid in concentrations of at least about 10 mg/ml. In various embodiments, the CXCL12-interacting heparinoid is present in a concentration of at least about 15 mg/ml, at least about 20 mg/ml, at least about 30 mg/ml, at least about 40 mg/ml, at least about 50 mg/ml. In certain embodiments, the CXCL12-interacting heparinoid is packaged in sterile-filled 10 ml glass vials containing an isotonic 50 mg/ml solution of CXCL12-interacting heparinoid in buffered saline.

5.5.2. Pharmaceutical Compositions Formulated for S.C. Administration

In various embodiments, the pharmaceutical composition is formulated for subcutaneous administration.

In certain such embodiments, the CXCL12-interacting heparinoid is associated with multivalent cations. The term “associated”, when used to describe the relationship between a heparinoid and a cation, means a chemically relevant association. The association may be as a salt, ion/counterion, complex, binding, coordination or any other chemically relevant association. The exact nature of the association will be readily apparent to a person of skill in the art depending on the form of the composition.

In various such embodiments, the multivalent cations are selected from cations having a charge of +2, +3, +4, or greater. In some embodiments, the multivalent cation is an ion that contains both positive and negative charges, with a net charge greater than +1. Exemplary multivalent cations include metal ions, amino acids, and other organic and inorganic cations. In certain embodiments, the ion is a metal ion that is Zn²⁺, Ca²⁺, Mg²⁺ or Fe²⁺. In a specific embodiment, the cation is Ca²⁺. In another specific embodiment, the cation is Mg²⁺.

In certain of the embodiments of pharmaceutical composition intended for subcutaneous administration, the CXCL12-interacting heparinoid is associated primarily with one species of multivalent cation. In other embodiments, the CXCL12-interacting heparinoid is associated with several different multivalent cation species. In specific embodiments, the CXCL12-interacting heparinoid is associated with Mg²⁺ and Ca²⁺.

In the multivalent cation embodiments, multivalent cations may be introduced to the CXCL12-interacting heparinoid composition at any step.

In one embodiment, the CXCL12-interacting heparinoid is substantially desulfated at the 2-O and 3-O positions, and the multivalent cation is present during alkaline hydrolysis of the heparin starting material. In certain embodiments, the multivalent cation is present as the chloride salt. In certain embodiments, the multivalent cation is present as the hydroxide salt. In one embodiment, the chloride salt is preferred for use during solution phase alkaline hydrolysis. In another embodiment, the hydroxide salt is preferred for use during solid phase alkaline hydrolysis. In another embodiment, the hydroxide salt is preferred for use when alkaline hydrolysis is performed as a paste. Certain multivalent cations may affect the level of desulfation if present during alkaline hydrolysis, and may be used to achieve desired levels of desulfation. The amount of the multivalent cation may be titrated to control the amount of desulfation as described in U.S. Pat. No. 5,296,471 at Example 4 therein.

Thus, when a multivalent cation is used during alkaline hydrolysis, the multivalent cation concentration used should be adjusted based on both the desired level of desulfation and the desired concentration of the final product. The molar multivalent cation concentration used during alkaline hydrolysis may be substantially less than the molar heparin concentration. Preferably, the molar ratio (multivalent cation:heparin) is about 0.1, 0.2, 0.3, 0.4, 0.5, 0.6, 0.7, 0.8, 0.9, 1.0, 1.1, 1.2, 1.3, 1.4, or 1.5, or any ranges composed of those values. Preferably, the concentration of the multivalent cation used during alkaline hydrolysis is about 0.01 mM, 0.05 mM, 0.1 mM, 0.5 mM, 1 mM, 5 mM, 10 mM, 20 mM, 50 mM, 100 mM, 250 mM, 500 mM or 1M or any range composed of those numbers.

In certain embodiments, primarily monovalent cations are present during the cold alkaline hydrolysis step, and the multivalent cation is added later, during reconstitution of the lyophilate. In a most preferred embodiment, either MgCl₂ or CaCl₂ is added at high concentration during reconstitution of the lyophilate.

The multivalent cation concentration used during reconstitution may be equal to the concentration of the cation used during alkaline hydrolysis. Preferably, the multivalent cation concentration is at least about 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold the concentration of the cation used during alkaline hydrolysis. Preferably, the concentration of the multivalent cation used during reconstitution is about 0.1 M, 0.5 M, 1 M, 2 M, 3 M, 4 M, 5M, or greater. Most preferably, the concentration is about 2 M.

Excess cations can be removed by any method known to those in the art. One preferred method of removing excess cations is the use of a desalting column. Another preferred method of removing excess cations is dialysis. After removal of excess ions, the solution preferably has about equal, 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold greater multivalent cation concentration to monovalent cation concentration. The solution may also be free or substantially free of monovalent cations.

In typical embodiments, the final concentration of CXCL12-interacting heparinoid in the pharmaceutical composition is between 0.1 mg/mL and 600 mg/mL. In certain embodiments, the final concentration of partially desulfated heparin in the pharmaceutical composition is between 200 mg/mL and 400 mg/mL.

In some embodiments, the concentration of heparinoid is greater than about 25 mg/mL. In certain embodiments, the concentration of heparinoid is greater than about 50 mg/mL. In a variety of embodiments, the concentration of heparinoid is greater than about 60 mg/mL, 70 mg/mL, 80 mg/mL, 90 mg/mL, or 100 mg/mL.

In specific embodiments, the CXCL12-interacting heparinoid is present in the pharmaceutical composition in a concentration greater than about 110 mg/mL, 120 mg/mL, 130 mg/mL, 140 mg/mL, 150 mg/mL, 160 mg/mL, 170 mg/mL, 180 mg/mL, or even greater than about 190 mg/mL or 200 mg/mL. In specific embodiments, the CXCL12-interacting heparinoid is present in the pharmaceutical composition at a concentration of about 175 mg/mL. In another embodiment, the CXCL12-interacting heparinoid is present in the pharmaceutical composition at a concentration of about 200 mg/mL. In one embodiment, the CXCL12-interacting heparinoid is present in the pharmaceutical composition at a concentration of 400 mg/mL.

In certain embodiments, the concentration of CXCL12-interacting heparinoid is 50 mg/mL to 500 mg/mL, 100 mg/mL to 400 mg/mL, or 150 mg/mL to 300 mg/mL. In specific embodiments, the concentration is 50 mg/mL, 100 mg/mL, 150 mg/mL, 200 mg/mL, 250 mg/mL, 300 mg/mL, 350 mg/mL, 400 mg/mL, 450 mg/mL or 500 mg/mL. In certain currently preferred embodiments, the concentration is 200 mg/mL, 300 mg/mL or 400 mg/mL.

In typical embodiments, the pharmaceutical composition has a viscosity of less than about 100 cP. In various embodiments, the pharmaceutical composition has a viscosity of less than about 80 cP. In certain embodiments, the pharmaceutical composition has a viscosity of less than about 60 cP. In particular embodiments, the pharmaceutical composition has a viscosity of less than about 20 cP.

In typical embodiments, the pharmaceutical composition has an osmolality less than about 2500 mOsm/kg. In various embodiments, the pharmaceutical composition has an osmolality between about 150 mOsm/kg and about 500 mOsm/kg. In certain embodiments, the pharmaceutical composition has an osmolality between about 275 mOsm/kg and about 300 mOsm/kg. In a particular embodiment, the pharmaceutical composition has an osmolality of about 285 mOsm/kg. In a specific embodiment, the pharmaceutical composition is isotonic.

### 6. EXAMPLES

Practice of the various embodiments of the methods can be understood through reference to the following examples, which are provided by way of illustration and are not intended to be limiting.

### 6.1. Example 1

### ODSH Attenuates Myelosuppressive Side Effects of Induction Chemotherapy in Treatment of Acute Myeloid Leukemia

A single arm open-label clinical study was conducted at multiple trial sites (University of Utah, Georgia Regents University, and Medical University of South Carolina) in patients with newly diagnosed acute myeloid leukemia (AML), seeking to confirm that ODSH could accelerate platelet and white blood cell (WBC) recovery in patients receiving induction chemotherapy with a regimen known to have myelosuppressive side effects.

All patients received the following standard (“7+3”) induction regimen,
Idarubicin (12 mg/m²/day) by short intravenous infusion on Days 1, 2, and 3; and
Cytarabine (100 mg/m²) as a continuous intravenous infusion over 24 hours (Days 1 through 7).

All patients also received ODSH as an intravenous bolus immediately after the idarubicin dose on Day 1, at a dose of 4 mg/kg, followed by a continuous intravenous infusion at a dose of 0.25 mg/kg/hr for a total of 7 days (Days 1 through 7).

ODSH was manufactured under cGMP conditions by Scientific Protein Labs (Waunakee, Wis.) by cold alkaline hydrolysis of USP porcine intestinal unfractionated heparin during lyophilization. This process removes 2-O and 3-O sulfates, leaving N- and 6-O sulfates and carboxylates intact (Fryer et al., J. Pharmacol. Exp. Ther. 282:208-2219 (1997)). Seven serial 1.2 kg batches of material have shown an average molecular mass of 11.7±0.3 kDa, low affinity for anti-thrombin III (Kd=339 μm, or 4 mg/ml) (vs. 1.56 μm or 22 μg/ml for UFH), and consistently reduced USP anticoagulant activity (7±0.3 U of anticoagulant activity/mg), anti-Xa activity (1.9±0.1 U/mg), and anti-Ha activity (1.2±0.1 U/mg) as compared with those of heparin (165-190 U/mg activity for all 3 assays). Drug product was formulated by Pyramid Laboratories (Costa Mesa, Calif.) in sterile-filled 10 ml glass vials containing an isotonic 50 mg/ml solution of sodium ODSH in buffered saline.

Twelve patients were enrolled. The median age was 56 (range 22-74). Based on cytogenetic, molecular, or antecedent hematologic disorder, 9 of 12 patients fell into the intermediate or poor risk categories. Patients did not receive growth factor support, that is, Neupogen or similar agents, during induction cycles. Complete remission at the end of the induction cycle was assessed using International Working Group (IWG) criteria (see, e.g., Cheson et al., J. Clin. Oncol. 21:4642 (2003)).

Platelet, neutrophil, and WBC recovery of the ODSH-treated AML patients was compared with the recovery in historical control patients receiving identical doses of idarubicin and cytarabine as induction therapy in a previous clinical study comparing idarubicin and daunorubicin in combination with cytarabine (Vogler et al., J. Clin. Oncol. 10(7):1103-11 (1992)). In the previous study, 101 patients received idarubicin 12 mg/m² on days 1, 2, and 3 and cytarabine 100 mg/m² on days 1-7. Growth factors were not administered to any patient.

Table 3 compares hematologic recovery parameters as reported in the prior study to those observed in the current study in which patients additionally received ODSH, as described above.

TABLE 3

| | Idarubicin + | Idarubicin + |
|---|---|---|
| | Cytarabine | Cytarabine + ODSH |
| | (n = 101) | (n = 12) |
| Time to platelet | 35 days | 22 days |
| count >50,000 | | |
| Time to WBC >1000 | 31 days | 21 days |

In the current study, 11 out of 12 patients (92%), including two patients who received an incomplete course of chemotherapy (3 and 5 days, respectively), had a morphologic complete remission by IWG criteria at the end of a single induction cycle. The only patient who did not obtain a complete morphologic remission at the end of induction therapy presented with extensive mediastinal and peripheral lymphadenopathy involved with granulocytic sarcomas, accompanying bone marrow involvement with AML. This patient had residual extramedullary disease at the end of his induction cycle, and achieved a complete remission with a subsequent cycle of FLAG-Ida chemotherapy without ODSH.

With 9 of 12 patients having intermediate or poor risk disease prior to treatment, and with two of these patients having received an incomplete course of treatment, the 92% complete remission rate after the first induction cycle is higher than would otherwise be expected based on historical data, as shown in Table 4.

TABLE 4

| | Idarubicin + | Idarubicin + |
|---|---|---|
| | Cytarabine | Cytarabine + ODSH |
| | (n = 101) | (n = 12) |
| Complete response with | 58% | 92% |
| first induction cycle | | |

Furthermore, 10 of the 12 patients remain in complete remission 5-13 months after having been enrolled in the study.

### Post-Induction Treatment and Outcomes

Of the 12 patients enrolled, 4 were not eligible to receive post-induction treatment on study due to either age≧60 years, induction failure, or incomplete induction. Of the other patients, one developed a line-associated deep venous thrombosis requiring systemic anticoagulation and was taken off study before consolidation. The remaining 4 patients each received one or more cycles of HIDAC and CX-01 consolidation treatment on study as follows: Patient 1005 completed all four cycles on study; Patient 1009 received 3 cycles of consolidation on study and asked to be taken off study before receiving the fourth cycle of HIDAC consolidation; Patient 3001 completed 1 consolidation cycle, withdrew from study and was lost to follow-up; and Patient 3003 received 2 cycles of consolidation on study before relapsing. Four patients who completed induction received an allogeneic stem cell transplant in CR1 (Patients 1002, 1006, 1010, and 3002). Six patients relapsed at a median time of 8 months. Among those were Patient 2001 who had not completed induction and relapsed 7 weeks after diagnosis and Patient 3001 who received only 1 cycle of consolidation therapy, and relapsed 13.5 months after diagnosis.

With a median follow-up of 14.2 months, median event free survival is 13.5 months and median OS is 13.6+ months.

### 6.2. Example 2

### ODSH Mobilizes Cells of Multiple Lineages from Bone Marrow

Bone marrow biopsies were obtained in the AML trial described in Example 1.

FIGS. 2A-2C are photomicrographs of biopsies from one of the patients. FIG. 2A is a photomicrograph prior to treatment, and shows the bone marrow packed with leukemia cells. FIG. 2B is a photograph of bone marrow at day 14 of the induction cycle, showing elimination of leukemia cells, as expected, and showing additionally an unexpected and significant depletion of normal bone marrow cells. FIG. 2C shows the bone marrow at Day 28, showing no evidence of leukemia and restoration of normal bone marrow appearance and function.

The unexpected clearing of the marrow seen in the Day 14 marrow suggests that the increased remission rate observed in the current trial can be attributed to ODSH-mediated mobilization of leukemic cells from the marrow into the peripheral circulation, where they became vulnerable to the infusions of cytarabine and idarubicin. Retention of leukemic cells in the bone marrow is known to make them more resistant to chemotherapy (Hope et al., Nat. Immunol. 5:738-742 (2004)).

The recovery by Day 28 demonstrates further that the ODSH-mediated flushing of cells from the marrow does not adversely affect the ability of the marrow to repopulate and support multi-lineage hematopoiesis. Indeed, the accelerated recovery of platelet and white cell count, consistent with observations from a previous trial in pancreatic cancer, demonstrates that the marrow microenvironments required for thrombopoiesis, erythropoiesis, and granulopoiesis remain healthy.

### 6.3. Example 3

### ODSH Inhibits CXCL12 Binding to CXCR4

CXCL12, also known as Stromal Cell Derived Factor-1 or SDF-1, was originally described as a CXC chemokine produced locally within the bone marrow compartment to provide a homing signal for hematopoietic stem cells (“HSC”s). CXCL12 is the ligand for the CXCR4 receptor on the surface of HSCs; ligation of CXCR4 by CXCL12 is known to promote stem cell survival, proliferation, migration, and chemotaxis (see, e.g., Lapidot et al., Leukemia 16(10):1992-2003 (2002)). It has also been reported that the CXCR4 receptor is prominently expressed on the cell membrane of many cancer cells, particularly cancer stem cells (Yu et al., Gene 374:174-9 (2006); Cojoc et al., Oncotargets & Therapy 6:1347-1361 (2013)), and that the CXCL12/CXCR4 interaction may mediate migration of cancer cells to anatomic sites that produce CXCL12 (Wald et al., Theranostics 3:26-33 (2013); Cojoc et al., supra).

To determine whether the ODSH-mediated mobilization of cells from the bone marrow observed in Example 2 was attributable to abrogation of or interference with the binding of CXCL12 to CXCR4, an in vitro inhibition assay was performed.

Polyvinyl 96-well high bind microplates (Corning Life Sciences, Corning, N.Y.) were coated with 0.5 μg/well of recombinant human CXCL12 (R&D Systems, Minneapolis, Minn.). Plates were incubated overnight at 4° C. and washed three times with PBS-0.05% Tween-20 (PBST). Separately, a constant amount of recombinant CXCR4 (Abnova, Taipei, Taiwan, 100 μL containing 0.8 μg/mL in PBST-0.1% BSA) was incubated with an equal volume of serially diluted ODSH (0.001-1,000 μg/mL in PBST-BSA) overnight at 4° C. The following day, 50 μL of CXCR4-ODSH mix was transferred to each respective CXCL12-coated well and incubated at 37° C. for 2 h. Wells were then washed four times with PBST. To detect bound CXCR4, 50 μL of a mouse anti-human CXCR4 antibody (R&D Systems, Minneapolis, Minn.) (1 μg/mL, in PBST) was added to each well, the mixture was incubated for 1 h at room temperature, and the wells were washed again four times with PBST. Horse-radish peroxidase-conjugated secondary antibody (R&D Systems, Minneapolis, Minn.) (50 μL per well) was added, wells were incubated for 1 h at room temperature, and then washed once with PBST. A colorimetric reaction as initiated by addition of 50 μL of tetramethyl benzidine chromogen (TMB) single solution substrate (Life Technologies, Frederick, Md.) and terminated after 15 min by addition of 50 μL of 1N HCl. Absorbance at 450 nm was read using an automated microplate reader. IC₅₀ values were determined from the plot of absorbance values vs. concentrations of ODSH.

As shown in FIG. 3, ODSH inhibits binding of CXCL12 (SDF-1) to CXCR4 in a concentration-dependent fashion, with an IC₅₀ of 0.010 μg/ml. This inhibitory concentration is well within the range of plasma concentrations expected to have been achieved in the AML trial: as detailed in Example 2, patients were administered a bolus of 4 mg/kg followed by a continuous intravenous infusion at a dose of 0.25 mg/kg/hr for a total of 7 days; an earlier phase I study had demonstrated that a bolus of 8 mg/kg followed by continuous intravenous infusion of 0.64 to 1.39 mg/kg/h provides a maximum mean plasma level of about 170 μg/ml, and steady state concentrations of about 40 μg/mL (Rao et al., Am. J. Physiol. Cell Physiol. 299:C997-C110 (2010)).

Any heparin derivative that is capable of inhibiting, reducing, abrogating, or otherwise interfering with, the binding of CXCL12 to CXCR4, such as those capable of binding to CXCL12 and/or CXCR4 and preventing binding of CXCL12 to CXCR4, and that can safely be given at concentrations that reduce the binding of CXCL12 to CXCR4 without significant anticoagulation, should be useful in mobilizing HSCs.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of obtaining hematopoietic stem cells (HSC) from a donor, comprising:
administering to a donor subject a CXCL12-interacting heparinoid in an amount effective to mobilize HSC cells; and
isolating the HSC cells from the peripheral blood of the donor.

2-4. (canceled)

5. The method of, claim 1, wherein
the CXCL12-interacting heparinoid is administered to the donor subject at least about 1 hr to about 24 hr prior to isolating the HSC cells.

6. The method of claim 1, further comprising adjunctively administering in combination with the CXCL12-interacting heparinoid a bone marrow cell mobilizing agent.

7. The method of claim 6, wherein the bone marrow cell mobilizing agent is selected from the group consisting of G-CSF, GM-CSF, plerixafor, cyclophosphamide, 5-fluorouracil, and BIO5192.

8-11. (canceled)

12. The method of claim 1, wherein the CXCL12-interacting heparinoid is substantially desulfated at the 2-O position and/or 3-O position.

13. (canceled)

14. The method of claim 12, wherein the CXCL12-interacting heparinoid is at least 85% desulfated at the 2-O.

15. The method of claim 12, wherein the CXCL12-interacting heparinoid is at least 85% desulfated at the 3-O position.

16. The method of claim 12, wherein the CXCL12-interacting heparinoid is at least 85% desulfated at each of the 2-O and 3-O positions.

17. (canceled)

18. The method of claim 12, wherein the CXCL12-interacting heparinoid has an average molecular weight of about 8 kDa to about 15 kDa.

19-24. (canceled)

25. The method of claim 1, wherein the CXCL12-interacting heparinoid is administered intravenously.

26-28. (canceled)

29. The method of claim 1, wherein the CXCL12-interacting heparinoid is administered subcutaneously.

30. (canceled)

31. A method of hematopoietic stem cell (HSC) transplantation, comprising:
(a) administering to a HSC donor an amount of a CXCL12-interacting heparinoid effective to mobilize HSC cells in the bone marrow;
(b) isolating the HSC cells from the donor; and
(c) transplanting the HSC cells into a recipient subject in need of HSC transplantation.

32. (canceled)

33. The method of claim 31, wherein the donor is an autologous donor and the isolated HSCs are further treated to remove disease cells.

34. The method of claim 31, wherein the isolated HSCs are recombinantly engineered prior to transplantation.

35. The method of claim 31, wherein the recipient subject is treated with a myeloablative, reduced intensity myeloablative, or non-myeloablative treatment regimen.

36. The method of claim 31, wherein the donor is an autologous donor for autologous HSC transplantation, and the subject in need of HSC transplantation is diagnosed with a clinical indication selected from the group consisting of multiple myeloma, non-Hodgkin's lymphoma, Hodgkin's disease, acute myeloid leukemia, neuroblastoma, amyloidosis, neuroblastoma, systemic lupus erythematosus (SLE), systemic sclerosis, germ cell tumors (ovarian cancer), breast cancer, prostate cancer, lung cancer, colon cancer, skin cancer, liver cancer, pancreatic cancer, and sarcoma.

37. The method of claim 31, wherein the donor is an allogeneic donor for allogeneic HSC transplantation, and wherein the recipient subject in need of HSC transplantation is diagnosed with a clinical indication selected from the group consisting of acute myeloid leukemia, acute lymphoblastic leukemia, chronic myeloid leukemia, chronic lymphocytic leukemia, myeloproliferative disorders, myelodysplastic syndromes, multiple myeloma, Non-Hodgkin lymphoma, Hodgkin disease, aplastic anemia, pure red-cell aplasia, paroxysmal nocturnal hemoglobinuria, Fanconi anemia, thalassemia major, sickle cell anemia, severe combined immunodeficiency (SCID), Wiskott-Aldrich syndrome, hemophagocytic lymphohistiocytosis, inborn errors of metabolism, epidermolysis bullosa, severe congenital neutropenia, Shwachman-Diamond syndrome, Diamond-Blackfan anemia, leukocyte adhesion deficiency, and HIV infection.

38. A method of improving hematopoietic stem cell (HSC) engraftment in an HSC transplant recipient, comprising:
administering to a recipient subject in need of HSC transplantation a CXCL12-interacting heparinoid in an amount effective to enhance engraftment of graft HSC.

39. The method of claim 38, further comprising treating the subject with a reduced intensity or non-myeloablative conditioning regimen prior to transplanting graft HSCs.

40-43. (canceled)

44. The method of claim 38, wherein the subject in need of HSC transplantation is diagnosed with a condition selected from the group consisting of multiple myeloma, non-Hodgkin's lymphoma, Hodgkin's disease, acute myeloid leukemia, neuroblastoma, amyloidosis, neuroblastoma, systemic lupus erythematosus (SLE), systemic sclerosis, germ cell tumors (ovarian cancer), breast cancer, prostate cancer, lung cancer, colon cancer, skin cancer, liver cancer, pancreatic cancer, and sarcoma.

45-52. (canceled)

53. The method of claim 38, wherein the CXCL12-interacting heparinoid is substantially desulfated at the 2-O position and/or 3-O position.

54. (canceled)

55. The method of claim 53, wherein the CXCL12-interacting heparinoid is at least 85% desulfated at the 2-O position.

56. The method of claim 53, wherein the CXCL12-interacting heparinoid is at least 85% desulfated at the 3-O position.

57. The method of claim 53, wherein the CXCL12-interacting heparinoid is at least 85% desulfated at each of the 2-O and 3-O positions.

58-102. (canceled)

103. A method of obtaining T lymphocytes for T cell immunotherapy, comprising:
administering to a donor a CXCL12-interacting heparinoid in an amount effective to increase in the peripheral blood of the donor the concentration of T lymphocytes that have at least one desired phenotype; and
obtaining T lymphocytes from the donor's peripheral blood.

104. The method of claim 103, wherein the at least one desired phenotype is selected from the group consisting of:
CD4⁺;
CD8⁺;
CD45RA⁺, CD62L⁺, CD28⁺, CD95⁻;
CD45RA⁺, CD62L⁺, CD28⁺, CD95⁺;
CD45RO⁺, CD62L⁺, CD28⁺, CD95⁺;
CD45RO⁺, CD62L⁻, CD28⁺;
CD45RO⁺, CD62L⁻, CD28⁺, CD95⁺;
CD45RO⁺, CD62L⁻, CD28⁺, Perforin^{hi}, Gzmb^{hi}; and
CD45RO⁺, CD62L⁻, CD28⁺, CD95⁺, Perforin^{hi}, Gzmb^{hi}.

105. The method of claim 103, wherein the T lymphocytes so obtained are transduced ex vivo with at least one expression vector.

106. The method of claim 105, wherein the vector drives expression of a chimeric antigen receptor (CAR).

107. The method of claim 105, wherein the vector drives expression of at least a portion of a T cell receptor (TCR).

108. The method of claim 103, further comprising the subsequent step of:
administering the donor T lymphocytes to a recipient.

109. The method of claim 108, wherein the recipient is the same individual as the donor.

110-113. (canceled)

114. The method of claim 103, wherein the CXCL12-interacting heparinoid is substantially desulfated at the 2-O position and/or 3-O position.

115-133. (canceled)

134. A method of conditioning a T cell immunotherapy recipient to enhance the establishment or grafting of donor T cells in the recipient, comprising:
administering a CXCL12-interacting heparinoid to the recipient in an amount and for a period sufficient to deplete the recipient's bone marrow of cellular elements, at a time when depleting the recipient's bone marrow of cellular elements enhances establishment or grafting of donor T lymphocytes in the recipient.

135. The method of claim 134, wherein the CXCL12-interacting heparinoid is administered prior to administration of the T lymphocytes.

136. The method of claim 135, wherein the CXCL12-interacting heparinoid is administered prior to and during the time of T cell administration.

137-140. (canceled)

141. The method of claim 134, wherein the CXCL12-interacting heparinoid is substantially desulfated at the 2-O position and/or 3-O position.

142-166. (canceled)

167. The method of claim 1, wherein the isolated HSC cells are purified or substantially purified after isolation.

168. The method of claim 167, wherein the purification or substantial purification of the isolated HSC cells is carried out by distinguishing the presence or absence of one or more cell surface markers on the HSC cells.

169. The method of claim 168, wherein the cell surface markers comprise one or more of CD34, CD38, CD90, CD133, CD105, CD45, c-kit, sca-1, CD150, CD48, CD44, CD16 and CD32.

170-180. (canceled)
